(19) United States
(12) Patent Application Publication
(10) Pub. No.: US 2024/0159737 A1
(43) Pub. Date: May 16, 2024

(54) CELLULAR SELECTIVITY PROFILING AGAINST RNA HELICASES AND SPLICING REGULATORS

(71) Applicant: Baylor College of Medicine, Houston, TX (US)

(72) Inventors: Thomas F. Westbrook, Houston, TX (US); Kristen Karlin, Houston, TX (US); Calla Olson, Houston, TX (US)

(21) Appl. No.: 18/549,772

(22) PCT Filed: Mar. 8, 2022
(86) PCT No.: PCT/US2022/071040

§ 371 Date: Sep. 8, 2023

Related U.S. Application Data
(60) Provisional application No. 63/158,725, filed on Mar. 9, 2021.

(51) Int. Cl. G01N 33/50

(52) U.S. Cl. CPC G01N 33/5023; G01N 33/5011; G01N 33/5038

(57) ABSTRACT

Embodiments of the disclosure include chemical, genetic, and/or computational systems, methods, and compositions for characterizing RNA helicases for targeting for inhibition, and methods of screening for inhibitors. In specific embodiments, inhibitors of RNA helicases, including RNA helicases for splicing, are identified that have selective targeting of one or more desired RNA helicases but that are also counter-selected such that the inhibitor does not target one or more RNA helicases that would result in toxicity.

This application claims priority to U.S. Provisional Patent Application Ser. No. 63/158,725, filed Mar. 9, 2021, which is incorporated by reference herein in its entirety.

### TECHNICAL FIELD

Embodiments of the disclosure include at least the fields of cell biology, molecular biology, RNA metabolism, drug design and development, drug screening, and medicine.

### BACKGROUND

RNA helicases are ATPases that participate in most aspects of RNA metabolism and processing. Recent functional genomic studies (RNAi and CRISPR) have revealed that cancer cells are uniquely dependent on RNA helicases for survival. Notably, dependency on a specific RNA helicase often varies across tumor type and genetic background, suggesting selective dependency across tissue-of-origin and/or associated oncogenic drivers. However, the mechanisms driving these tissue- and/or genotype-specific dependencies remain elusive in part due to a lack of suitably selective and potent tool compounds to systematically explore the role of RNA helicases in cancer.

### BRIEF SUMMARY

Embodiments of the disclosure include methods and compositions that allow for drug discovery programs related to RNA metabolism, including at least being related to synthesis, folding/unfolding, modification, processing and degradation of RNA. In specific embodiments, the methods and compositions allow for drug discovery programs related to RNA processing including any modification made to RNA between its transcription and its final cellular function, such as at least RNA splicing. In some embodiments the methods and compositions concern RNA helicases and/or splicing regulators as the subject of characterization and selectivity profiling, and whether or not the RNA helicases are involved in splicing. In at least some embodiments, the methods provide for selectivity profiling of inhibitors for RNA splicing and include splicing regulators that are not themselves RNA helicases.

Methods and compositions of the disclosure allow for characterization of selectivity of components of RNA metabolism including development of assays that typify the components such that inhibitors are sufficiently selective for their intended purpose. In particular embodiments, the disclosure encompasses RNA helicase and/or splicing regulator drug discovery systems, including by utilizing a cellular platform and, at least in some cases, also a computational platform comprising protein(s) for which an inhibitor is desired and one or more assays to monitor the in-cell or in vivo activity of the protein. In specific embodiments, the protein to be targeted is an enzyme, and the protein may be a regulator (including an enzyme, in at least some cases) of splicing; in specific cases the protein is a component (whether permanent or transient) of the spliceosome. In specific embodiments, the disclosure encompasses use of a cellular and (optionally) computational platform comprising cell line or mouse models for selective degradation of RNA helicase or other proteins of interest and assays to monitor their in-cell or in vivo activity. Systems of the disclosure provide the assays required to evaluate target RNA helicase compound selectivity across a broader RNA helicase family.

Although in some cases the methods concern selectivity profiling of RNA helicases that are not involved in splicing, in specific embodiments the methods concern selectivity profiling of inhibitors for RNA helicases that are involved in splicing or for other components related to splicing, such as splicing regulators of any kind. These splicing-related RNA helicases and other components may be directly involved in processing of the RNA such that they may be associated with cancer cells when they are defective, in some cases. As such, at least some methods of the disclosure provide for identification of inhibitors that target these splicing-related RNA helicases or splicing regulators for cancer cells. At the cellular level, in some embodiments use of the inhibitors may reduce the amount of misprocessed RNAs that had accumulated as a result of defective splicing component(s), including with neurodegeneration or other disease indications. Inhibition of one or more targets that would result in a reduction of the misprocessed RNA accumulation may then be therapeutic for the cell or individual. In some cases, use of the inhibitors may increase the amount of misprocessed RNAs that had accumulated as a result of defective splicing component(s), such as in cancer indications. The disclosure encompasses development of inhibitors to components that regulate splicing and ensure that splicing occurs properly, such that inhibitors for those targets may increase the amount of misprocessed RNA. In specific settings, such as in cancer, this is desirable because the accumulation of misprocessed RNA directly or indirectly results in cell death. In specific embodiments, cancer cells are pre-disposed to have a dependency on RNA helicases/splicing regulators such that splicing becomes aberrant more easily (e.g., there is less inhibition of a particular target than may be required for a normal cell). In particular cases, accumulation of those misprocessed RNAs leads to immune signaling or other signaling events that result in cell death. In any embodiment, though, the selectivity of methods of the disclosure allows for identification of these inhibitors such that they also do not target RNA helicases or other components whose inhibition would be toxic to cells or an organism.

In particular embodiments, the disclosure provides improvements over methods in the art related to drug discovery of any kind by focusing evaluation of a particular inhibitor candidate with respect to a plurality of proteins, including a plurality of proteins of the same type or mechanism of action, but also with respect to ensuring counter selection against one or a plurality of proteins that may or may not be of the same type or mechanism of action. In a certain embodiment, the methods evaluate a particular inhibitor candidate with respect to a plurality of proteins involved in RNA metabolism but also that includes counter-selection against one or a plurality of proteins that are involved in RNA metabolism. In a specific embodiment, in-cell or in vivo methods evaluate a particular inhibitor candidate with respect to RNA helicases or splicing regulators but also allowing counter-selectivity against RNA helicases or splicing regulators that are not desired to inhibited by the inhibitor candidate. Thus, the disclosure allows evaluation of compounds across diverse RNA helicases (“counter-selection”) to characterize and drive selectivity of small molecule inhibitors against a target RNA helicase of interest in vivo. The RNA helicases may or may not be involved in splicing, and in cases wherein the RNA helicase is not involved in splicing, in some cases it may be assessed by methods encompassed herein because they are structurally related to RNA helicases that are involved in splicing. In specific embodiments, the term “structurally related” refers to helicases that are of the same sub-family of helicases, e.g. DEAD-box or DEAH-box, RIG-I-like, Ski2-like, and SF1. The degree of similarity between subfamily members may be determined by the alignment score based on clustalw alignment, a pairwise based alignment that scores for the primary sequence similarity between two proteins.

The methods of the present disclosure include assays that provide an in-cell view of affected processes following inhibition of an RNA helicase or splicing regulator. Thus, in at least some cases, the disclosure provides means for providing a signature response upon inhibition of an RNA helicase or splicing regulator. Specifically, the methods encompassed herein include ways for measuring a cellular or subcellular outcome upon inhibition of an RNA helicase or splicing regulator, for example at the transcriptome level. As used herein, the transcriptome includes messenger RNA molecules expressed from genes in the cells whether or not they are correctly spliced. In at least some cases, the transcriptome includes misprocessed mRNAs comprising part or all of one or more introns. These methods, in at least some cases, inform development of subsequent screening or other assays to assist in downstream steps (including, for example, selection of substrates for assays for particular RNA helicases or inclusion of certain cell markers). Thus, in particular embodiments the disclosure includes a multi-stage process that encompasses one or a series of assays that provide information for selective targeting for inhibition for one or more particular RNA helicases or splicing regulators, including during Lead Identification and Lead Optimization phases of drug discovery. Thus, applications encompassed herein enable optimal development of screening assays and, at least in some cases, secondary assays of any kind during initial target feasibility phases of drug discovery and also optimization.

In particular embodiments, the present disclosure is directed to systems, methods, and compositions for analyzing compositions related to targeting RNA splicing and/or RNA helicases. Systems, methods, and compositions related to screening, characterization, and development of inhibitors of particular RNA splicing components, including at least RNA helicases, are encompassed herein.

Embodiments of the disclosure include systems and methods for identifying drug targets in RNA processing, including for RNA splicing. In at least some cases, the systems and methods allow for targeting global RNA mis-splicing related to medical conditions such as cancer, autoimmune disease, infectious disease, neurodegeneration and so forth. In at least some embodiments, the systems and methods provide novel discovery systems and methods for selective RNA helicase inhibitors or splicing regulator inhibitors. As an example, a particular selective inhibitor identified by methods and systems described herein will target a class of RNA helicases that are master regulators of RNA splicing. That is, in at least some cases, the targeted RNA helicases may be involved in splicing of more than one gene and its targeting with a drug addresses mis-splicing by the targeted RNA helicases in which the mis-splicing results directly or indirectly in a medical condition, such as cancer, autoimmune diseases, infectious disease, or neurodegeneration. Therefore, the identified drugs by these systems and methods provide defined patient indications in oncology, immuno-oncology, infectious disease, autoimmune diseases, and neurodegeneration, for example. In some embodiments, splicing occurs normally in a disease state but the spliceosome is rate limiting because of increased transcriptional burden. In such cases, the diseased cell is highly sensitive to partial inhibition of the spliceosome. As a result, an increase in misprocessing occurs upon helicase inhibition that leads to death of the diseased cell.

Particular embodiments of the disclosed systems and methods model the inhibition (e.g., by degradation) of individual RNA helicases across a diverse family of RNA helicases to (1) enable understanding of shared and selective functions (“mechanism of action”) for target RNAhelicase(s) of interest, (2) develop selectivity signatures and assays for assessing in-cell activity of inhibitors against target RNA helicase of interest, (3) drive identification of pharmacodynamic (PD) markers for target RNA helicase of interest, and (4) drive discovery of predictive biomarkers for patient selection for inhibitors against target RNA helicase of interest.

The present disclosure provides systems and methods to characterize RNA helicase mechanism of action (MOA) and to uncover how these mechanisms lead to specific cancer or other dependencies. In embodiments wherein the mechanisms encompass RNA splicing, the systems and methods may characterize associations with cancer, given that somatic mutations in the spliceosome and other splicing factors are prevalent in cancer.

The disclosure also addresses development of in-cell and in-tissue assays for selectivity and pharmacodynamics markers using a cellular platform that may comprise cancer and normal cell lines and animal models in which an RNA helicase target and/or splicing regulator target can be selectivity degraded. These embodiments enable modeling of selective RNA helicase inhibition and/or selective splicing regulator inhibition prior to generation of tool or clinical-grade compounds.

In particular embodiments, a computational platform comprising proprietary algorithms and methods to quantify global and event-level changes in RNA processing allows for characterization of RNA helicase MOA or splicing regulator MOA and analysis of the relationship to specific cancer dependencies or dependencies related to other medical conditions. The computational embodiments also provide for development of in-cell and in-tissue assays for selectivity for drug targeting and for pharmacodynamics markers. In specific embodiments, the systems and methods of the disclosure enable identification of pharmacodynamic markers and signatures of specific RNA helicases inhibition that can be utilized to quantify in-cell and in-tissue activity and selectivity of RNA helicase inhibitors.

Embodiments of the disclosure include methods of screening for a selective inhibitor of one or more RNA helicases or one or more splicing regulators, comprising: providing an in-cell or in vivo assay for disruption of one or more RNA helicases or one or more splicing regulators; measuring one or more RNA metabolism parameters (RNA splicing, RNA decay, RNA catabolism, RNA export, transcription, translation, rRNA biogenesis, RNA modification, or a combination thereof) and/or proteomics in the cell following said disruption; subjecting one or more candidate inhibitors (small molecules, proteins, peptides, nucleic acid, carbohydrate, or a combination thereof) to the in-cell or in vivo assay for each RNA helicase in a plurality of helicases for identification of candidate inhibitors that modify the one or more RNA metabolism parameters and/or proteomics for a first subset of RNA helicases and/or splicing regulators in the plurality but that do not modify the one or more RNA metabolism parameters and/or splicing regulators for a second subset of RNA helicases in the plurality. In some embodiments, the disruption is at the gene level or at the mRNA or protein level. When the disruption is at the protein level, the disruption may be carried out by enzymatic degradation (including by the proteasome), small molecule inhibition of enzymatic activity, allosteric small molecule inhibitor, or small molecule degrader. When the disruption is at the gene level or at the mRNA level, the disruption may be carried out by RNAi, siRNA, shRNA, ribozymes, CRISPRCas9, homologous recombination, site-specific nucleases, zinc fingers, or TALENs.

For the subjecting step, the method may comprise subjecting one or more candidate inhibitors to the in-cell assay for each RNA helicase in the plurality to identify candidate inhibitors that modify the one or more parameters of RNA metabolism or proteomics for a first subset of RNA helicases in the plurality, followed by identifying the absence of modifying the one or more parameters of RNA metabolism or proteomics for the second subset of RNA helicases in the plurality. In another case, the subjecting step may be further defined as: subjecting one or more candidate inhibitors to the in-cell assay for each RNA helicase in the plurality to identify candidate inhibitors that do not modify the one or more parameters of RNA metabolism or proteomics for the second subset of RNA helicases in the plurality, followed by identifying the presence of modifying for the one or more parameters of RNA metabolism or proteomics for the first subset of RNA helicases in the plurality. In another case, the subjecting step may be further defined as: subjecting one or more candidate inhibitors to the in-cell assay for each RNA helicase in the plurality to identify candidate inhibitors that modify the one or more parameters of RNA metabolism or proteomics for a first subset of RNA helicases in the plurality at substantially the same time as identifying the absence of modifying the one or more parameters of RNA metabolism or proteomics for the second subset of RNA helicases in the plurality. In any case, the subjecting step may or may not comprise high throughput screening.

In specific embodiments, the methods utilize cellular assays, and the cells of the assays may be normal cells (including not cancerous) or diseased cells, including cancer cells. When cancer cells are utilized, the cancerous cells may be breast cancer, non-small cell lung cancer, small cell lung cancer, colorectal cancer, subtypes of leukemia, subtypes of lymphoma, ovarian cancer, esophageal cancer, hepatic cell carcinoma, head and neck cancer, gastric cancer, pancreatic cancer, or bladder cancer.

Measuring steps for the methods may be of any kind and may measure double stranded RNA, mRNA processing, differential expression, differential splicing, global RNA processing fidelity, RNA catabolites, protein levels, RNA substrates, RNA binding motifs, or a combination thereof. In some cases, the measuring step comprises measuring intron splicing in one or more genes upon degradation of one or more RNA helicases. The measuring step may comprise measuring intron splicing in multiple genes upon degradation of multiple RNA helicases. In some cases, information from measuring intron splicing in multiple genes upon degradation of multiple RNA helicases results in identification of inhibitors that modify RNA splicing for a first subset of RNA helicases in the plurality but that do not modify RNA splicing for a second subset of RNA helicases in the plurality.

In any method encompassed herein, the method may further comprising the step of formulating one or more inhibitors in a pharmaceutically acceptable carrier.

In some embodiments, at least some of the RNA helicases in the first subset of RNA helicases are from the same sub-family of RNA helicases. The RNA helicases in the first subset of RNA helicases may or may not share the same function in RNA metabolism, including a function related to RNA splicing.

Any RNA helicase may be the subject of any method of the disclosure, including at least one, two, or more (or the majority, or all) of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

In some embodiments, identification of candidate inhibitors that modify the one or more RNA metabolism parameters for a first subset of RNA helicases in the plurality but that do not modify the one or more RNA metabolism parameters for a second subset of RNA helicases in the plurality is performed by a computer and may utilize an algorithm.

In one embodiment, there is a method of predicting toxicity of a compound for an individual, comprising the steps of: providing the compound to a non-diseased cell and identifying a first RNA signature for the cell; and comparing the first RNA signature to a second RNA signature associated with disruption of an RNA helicase or splicing regulator in a cell, wherein said disruption is toxic to a cell or organism, wherein when the first RNA signature and the second RNA signature are the same or are substantially the same, the compound is (or is predicted to be) toxic for the individual, and wherein when the first RNA signature and the second RNA signature are not the same or not substantially the same, the compound is not (or is not predicted to be) toxic for the individual. In specific cases, the method may be further defined as: comparing the first RNA signature to a plurality of other RNA signatures each respectively associated with disruption of an RNA helicase or a splicing regulator, wherein said disruption of a subset of the plurality of other RNA helicases and/or splicing regulators is toxic to a cell or organism; and wherein when the first RNA signature and the RNA signatures of the subset are the same or are substantially the same, the compound is toxic for the individual, and wherein when the first RNA signature and the RNA signatures of the subset are not the same or not substantially the same, the compound is not toxic for the individual. The non-diseased cell may be a non-cancerous cell of the same type with respect to cancer cells of interest, is a normal peripheral blood mononuclear cell, or is a lymphocyte. The RNA signature from a disruption of an RNA helicase or splicing regulator may comprise misprocessed RNA.

Any comparisons for any method herein may be performed by a computer, including with an algorithm.

Some methods may further comprise the step of producing the disruption of the RNA helicase or a splicing regulator, and the disruption may be at the gene level or at the mRNA or protein level.

In one embodiment, there is a method of screening for an activator of antiviral immune signaling and/or antitumor immune signaling, comprising the steps of: subjecting cancer cells or normal cells separately to a plurality of candidate activators of antiviral immune signaling and/or antitumor immune signaling; measuring which candidate activators produce an RNA signature comprising accumulation of misprocessed RNAs in the cells; and comparing output from the measuring step to RNA signatures produced from one or a plurality of disrupted RNA helicases and/or splicing regulators in normal cells, wherein when a candidate activator produces an RNA signature comprising accumulation of misprocessed RNAs that is the same as, or substantially the same as, an RNA signature from a disrupted RNA helicase and/or splicing regulator that produces misprocessed RNA that activates antiviral immune signaling and/or antitumor immune signaling, then the candidate activator is an activator of antiviral immune signaling and/or antitumor immune signaling. The subjecting step may comprise subjecting the plurality to a variety of types of cancer cells and comparing the output between two or more cancer types to RNA signatures produced from the plurality of disrupted RNA helicases and/or splicing regulators. In some embodiments, the activator is not known to be an inhibitor of an RNA helicase or a splicing regulator or is not an inhibitor of an RNA helicase or a splicing regulator. In some embodiments, the activator is provided in a therapeutically effective amount to an individual in need thereof, such as one with cancer or an infectious disease.

In one embodiment, there is a method of identifying inhibitors of antiviral immune signaling and/or antitumor immune signaling, comprising the steps of: subjecting candidate inhibitors to cells and measuring from the cells respective RNA signatures for misprocessed RNA; and comparing the RNA signatures from the cells in the subjecting step to one or more RNA signatures from cells each comprising disruption of an RNA helicase or a splicing regulator, wherein when an RNA signature produced in the cells upon subjecting them to a candidate inhibitor comprises a reduced amount of misprocessed RNA and/or comprises a reduced amount of particular misprocessed RNA molecules when compared to the RNA signatures from cells each comprising disruption of an RNA helicase or a splicing regulator, the candidate inhibitor is an inhibitor of antiviral immune signaling and/or antitumor immune signaling. In specific cases, the activator is provided in a therapeutically effective amount to an individual in need thereof, such as one with cancer or an infectious disease.

In one embodiment, there is a method of screening for an inhibitor of antiviral immune signaling and/or antitumor immune signaling, comprising the steps of: subjecting cancer cells or normal cells separately to a plurality of candidate inhibitors of antiviral immune signaling and/or antitumor immune signaling; measuring which candidate inhibitors produce an RNA signature comprising accumulation of misprocessed RNAs in the cells; and comparing output from the measuring step to RNA signatures produced from one or a plurality of disrupted RNA helicases and/or splicing regulators in normal cells, wherein when a candidate inhibitor produces an RNA signature comprising accumulation of misprocessed RNAs that is the same as, or substantially the same as, an RNA signature from a disrupted RNA helicase and/or splicing regulator that produces misprocessed RNA that activates antiviral immune signaling and/or antitumor immune signaling, then the candidate inhibitor is an inhibitor of antiviral immune signaling and/or antitumor immune signaling. The inhibitor may be provided in a therapeutically effective amount to an individual in need thereof, such as one with neurodegeneration or an autoimmune disease.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows a phylogenetic tree demonstrating the evolutionary relationships among RNA helicases based upon sequence similarity.

FIGS. 2A-2C show small molecule inhibitor-like dose control and kinetics. FIG. 2A illustrates a degradation assay for an exemplary RNA helicase, DHX15. FIG. 2B provides a western blot analysis demonstrating does-dependent degradation of a fusion of FK506 binding protein and DHX15, per the assay, whereas FIG. 2C is a time-dependent degradation representation.

FIG. 3 demonstrates a broad application of the degradation assay across a variety of RNA helicases in triple negative breast cancer (TNBC) cell lines.

FIGS. 4A-4H illustrates a variety of assays for RNA phenotype following degradation of one or more specific RNA helicases to ascertain its effect on the transcriptome. Assays include single cell dsRNA imaging (FIG. 4A); global changes to mRNA processing fidelity (FIG. 4B); differentially affected genes (expression or splicing)(FIG. 4C); individual substrate identification (eCLIP) (FIG. 4D); RNA decay and catabolism by mass spectrometry (FIG. 4E), proteomics (FIG. 4F), pathway analysis (FIG. 4G), and substrate recognition for primary and secondary motifs (FIG. 4H).

FIG. 5 demonstrates analysis of RNA helicase fingerprints using routine methods in the art (left) and a novel algorithm of the present disclosure (right) showing sensitivity for detection of RNA mis-splicing.

FIG. 6 demonstrates identification of fingerprints of selective inhibition of RNA helicases using two representative genes: ZNF384 and MAEA. The gene map illustrates one intron in the respective genes, and the representations below demonstrate the quality of RNA splicing upon degradation of DXH15 vs. degradation of DHX38.

FIG. 7 demonstrates the relative intron retention for the SNAPC1 gene (as an example) in cell lines in which DHX16, DHX8, or DHX15 have been degraded.

FIG. 8 shows one embodiment for a system of selectivity profiling against a class of RNA helicases.

FIGS. 9A-9F show examples of decomposing read-pairs to learn RNA (mis)processing. FIG. 9A illustrates standard read alignment to properly processed RNA. FIG. 9B illustrates read alignment evidence of misprocessing utilized by routine method in the art. FIG. 9C illustrates additional read alignment evidence of misprocessing utilized by a novel algorithm of the present disclosure in addition to that utilized by routine method in the art. FIG. 9D describes read pair classification utilized by a novel algorithm of the present disclosure. E indicates exon, I indicates intron, IE indicates a read that aligns to an intron and exon. FIG. 9E illustrates the use of directed acyclic graph theory in a novel algorithm of the present disclosure. FIG. 9F illustrates typical read coverage for an example intron and shown in red are reads counted as introns (misprocessed RNA) in each method.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, a “disruption” of a gene or gene product in a cell refers either to (1) the elimination or reduction of expression of one or more gene products encoded by the subject gene in a cell, compared to the level of expression of the gene product in the absence of the disruption; or (2) the elimination or reduction of activity of one or more gene products. Exemplary gene products include mRNA and protein products encoded by the gene. Disruption in some assays is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene product disruption may be induced by artificial methods, such as physical inhibition of a protein (as the gene product) by a compound or by physical destruction of the protein, including degradation of the protein. The physical destruction of the protein may occur by any suitable method including at least enzymatic degradation, including marked for destruction by the proteosome. In some cases, the disruption of the protein occurs by enzymatic degradation, small molecule inhibition of enzymatic activity, allosteric small molecule inhibitor, or small molecule degrader (such as proteolysis targeting chimera (PROTAC) or molecular glue). Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques that result in targeted gene inactivation or disruption, e.g., by induction of breaks and/or homologous recombination. Examples of gene disruption methods include at least CRISPR/Cas9, homologous recombination, site-specific nucleases, zinc fingers, TALENs, and so forth. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination. In some cases, a gene or gene product is disrupted using small molecule antagonists.

As used herein, the term “pharmacodynamic or “PD” biomarker” refers to a biomarker whose level changes in response to exposure to an inhibitor (whether or not it is a test candidate inhibitor).

The term “platform” as used herein refers to cellular-associated assay(s) (in-cell or in vivo, etc.) related to inhibitor(s) and/or helicase(s) and/or splicing regulator(s) of interest. The total collection of assays, steps, stages, etc. may be used to determine the selectivity of one or more molecules. The nature of the assays, for example, may be determined by one or more factors, including helicase or splicing regulator of interest, inhibitor of interest, assay conditions, and so forth. In particular embodiments, the platform comprises the parallelized disruption of RNA helicases (in some cases, as well as other components of RNA splicing and/or other forms of RNA processing) for which the subsequent measurement of effects on RNA processing (e.g., through RNA sequencing methodologies) and the subsequent novel analysis methods reveal much greater sensitive of detection for RNA mis-processing and/or mis-splicing compared to current known methods (e.g., at least 30-100-fold). In at least some cases, other molecular, cellular, and computational approaches are also utilized in such a parallelized disruption platform. In any event the aggregate of assays in the platform provides input into a total “picture” of RNA mis-processing for given candidate RNA helicases or other proteins, including splicing regulators. In some embodiments herein, the platform includes computational embodiments to facilitate pattern recognition, including at least for RNA signatures following an event, such as disruption of a gene (including by the hand of man), exposure to a candidate modulator, and so forth.

As used herein, the term “RNA signature” refers to a single or combined collection of RNA molecules in a cell with a uniquely characteristic pattern of incidence and that may or may not be the result of an altered biological process (e.g., upon artificial and directed disruption of one or more RNA helicases or splicing regulators in the cell) or the result of a medical condition. The RNA signature may comprise part or all of a transcriptome (the messenger RNAs from a cell) and may or may not comprise other types of RNA, including rRNA and/or tRNA. In at least some cases, the RNA signature comprises mis-spliced RNAs that are the result of natural, normal errors in splicing and/or that are the result of errors in splicing as a result of one or more mutation events that may or may not be associated with a medical condition. The RNA signature may be the result of an event, such as disruption of a gene (including by the hand of man), exposure to a candidate modulator, and so forth. The content of the RNA signature may be determined by any suitable methods including at least next generation sequencing, as one example.

As used herein, the term “RNA splicing” refers to processing of RNA in which a newly made precursor messenger RNA transcript (that may be referred to as a “pre-mRNA”) is converted into a mature messenger RNA (mRNA). Such splicing includes removal of introns (non-coding regions) and linking of exons (coding regions). For many eukaryotic introns, a series of reactions catalyzed by the spliceosome produces the spliced mRNA.

As used herein, the term “selectivity” refers to the ability of an inhibitor to be able to inhibit the function of a desired component of RNA metabolism, such as an RNA helicase or splicing regulator, but wherein the inhibitor is not able to inhibit one or more other components of RNA metabolism, including one or more other RNA helicases or splicing regulators, respectively. In some cases, the selectivity comprises the ability to inhibit one or more particular, desired RNA helicases or splicing regulators but excludes the ability to inhibit one or more other RNA helicases or splicing regulators that would be toxic to a cell or organism if inhibited.

The term “splicing regulator” as used herein refers to any compound that directly or indirectly is associated with RNA splicing. The compound may be a protein, nucleic acid (e.g., small nuclear RNAs), and so forth. The splicing regulator may be a standing or transient component of the spliceosome. In particular cases, the splicing regulator (including when defective) is directly or indirectly associated with cancer, autoimmune disease, infectious disease, or neurodegeneration, such as the splicing regulator being in a defective state in cancer cells. In specific embodiments, the splicing regulator is one or more from the following list:

| Complex | Protein | Class/Family |
|---|---|---|
| Sm | SNRPB | Sm |
| Sm | SNRPD1 | Sm |
| Sm | SNRPD2 | Sm |
| Sm | SNRPD3 | Sm |
| Sm | SNRPE | Sm |
| Sm | SNRPF | Sm |
| Sm | SNRPG | Sm |
| U1 snRNP | RNU1-1 | U1 snRNP |
| U1 snRNP | SNRPA | U1 snRNP |
| U1 snRNP | SNRNP70 | U1 snRNP |
| U1 snRNP | SNRPC | U1 snRNP |
| U2 snRNP | RNU2-1 | 17S U2 snRNP |
| U2 snRNP | SNRPA1 | 17S U2 snRNP |
| U2 snRNP | SNRPB2 | 17S U2 snRNP |
| U2 snRNP | SF3B1 | 17S U2 snRNP |
| U2 snRNP | SF3B2 | 17S U2 snRNP |
| U2 snRNP | SF3B3 | 17S U2 snRNP |
| U2 snRNP | SF3B4 | 17S U2 snRNP |
| U2 snRNP | SF3B5 | 17S U2 snRNP |
| U2 snRNP | PHF5A | 17S U2 snRNP |
| U2 snRNP | SF3B6 | 17S U2 snRNP |
| U2 snRNP | SF3A1 | 17S U2 snRNP |
| U2 snRNP | SF3A2 | 17S U2 snRNP |
| U2 snRNP | SF3A3 | 17S U2 snRNP |
| U2 snRNP | DDX46 | 17S U2 snRNP associated |
| U2 snRNP | DDX39B | 17S U2 snRNP associated |
| U2 snRNP | HTATSF1 | 17S U2 snRNP associated |
| U5 snRNP | RNU5A-1 | U5 snRNP |
| U5 snRNP | SNRNP200 | U5 snRNP |
| U5 snRNP | PRPF8 | U5 snRNP |
| U5 snRNP | EFTUD2 | U5 snRNP |
| U5 snRNP | PRPF6 | U5 snRNP |
| U5 snRNP | DDX23 | U5 snRNP |
| U5 snRNP | CD2BP2 | U5 snRNP |
| U5 snRNP | SNRNP40 | U5 snRNP |
| U5 snRNP | TXNL4A | U5 snRNP |
| U4/U6 snRNP | RNU4-1 | U4/U6 snRNP |
| U4/U6 snRNP | RNU6-1 | U4/U6 snRNP |
| U4/U6 snRNP | PRPF4 | U4/U6 snRNP |
| U4/U6 snRNP | PRPF3 | U4/U6 snRNP |
| U4/U6 snRNP | PPIH | U4/U6 snRNP |
| U4/U6 snRNP | PRPF31 | U4/U6 snRNP |
| U4/U6 snRNP | NHP2L1 | U4/U6 snRNP |
| tri-snRNP | LSM2 | LSm |
| tri-snRNP | LSM3 | LSm |
| tri-snRNP | LSM4 | LSm |
| tri-snRNP | LSM5 | LSm |
| tri-snRNP | LSM6 | LSm |
| tri-snRNP | LSM7 | LSm |
| tri-snRNP | LSM8 | LSm |
| tri-snRNP | SART1 | tri-snRNP |
| tri-snRNP | USP39 | tri-snRNP |
| A complex | U2AF1 | 17S U2 snRNP associated |
| A complex | U2AF2 | 17S U2 snRNP associated |
| A complex | PUF60 | 17S U2 snRNP associated |
| A complex | SMNDC1 | 17S U2 snRNP associated |
| A complex | RBM17 | 17S U2 snRNP associated |
| A complex | U2SURP | 17S U2 snRNP associated |
| A complex | CHERP | 17S U2 snRNP associated |
| A complex | SF1 | A complex |
| A complex | PRPF40A | A complex |
| A complex | THRAP3 | A complex |
| A complex | RBM25 | A complex |
| A complex | CCAR1 | A complex |
| A complex | SUGP1 | A complex |
| A complex | RBM5 | A complex |
| A complex | RBM10 | A complex |
| A complex | HNRNPA1 | A complex |
| A complex | HNRNPAB | A complex |
| B complex | DHX15 | 17S U2 snRNP associated |
| PRP19 complex | PRPF19 | PRP19 complex |
| PRP19 complex | CDC5L | PRP19 complex |
| PRP19 complex | PLRG1 | PRP19 complex |
| PRP19 complex | CWC15 | PRP19 complex |
| PRP19 complex | BCAS2 | PRP19 complex |
| PRP19 complex | CTNNBL1 | PRP19 complex |
| PRP19 complex | WBP11 | PRP19 complex |
| PRP19 complex | PQBP1 | PRP19 complex |
| B complex | PPIE | PRP19 related |
| B complex | CRNKL1 | PRP19 related |
| B complex | SNW1 | PRP19 related |
| B complex | ISY1 | PRP19 related |
| B complex | XAB2 | PRP19 related |
| B complex | RBM22 | PRP19 related |
| B complex | PPIL1 | PRP19 related |
| B complex | BUD31 | PRP19 related |
| B complex | AQR | PRP19 related |
| B complex | SMU1 | B complex |
| B complex | MFAP1 | B complex |
| B complex | IK | B complex |
| B complex | WBP4 | B complex |
| B complex | TFIP11 | B complex |
| B complex | ZMAT2 | B complex |
| B complex | PRPF38A | B complex |
| B complex | PPIL4 | B complex |
| Bact complex | CWC27 | Bact complex |
| Bact complex | DHX16 | Bact complex |
| Bact complex | CWC22 | Bact complex |
| Bact complex | ZNF830 | Bact complex |
| Bact complex | CCDC12 | Bact complex |
| Bact complex | PPIL2 | Bact complex |
| Bact complex | GPKOW | Bact complex |
| Bact complex | RNF113A | Bact complex |
| Bact complex | PRCC | Bact complex |
| Bact complex | CWC25 | Bact complex |
| Bact complex | GPATCH1 | Bact complex |
| Bact complex | CDC40 | second step factor |
| Bact complex | BUD13 | RES complex |
| Bact complex | SNIP1 | RES complex |
| Bact complex | RBMX2 | RES complex |
| Bact complex | EIF4A3 | EJC |
| Bact complex | SAP18 | EJC |
| C complex | HSPA8 | PRP19 complex |
| C complex | SYF2 | C complex |
| C complex | DDX41 | C complex |
| C complex | CXorf56 | C complex |
| C complex | DGCR14 | C complex |
| C complex | C9orf78 | C complex |
| C complex | PPIL3 | C complex |
| C complex | PPWD1 | C complex |
| C complex | DHX35 | C complex |
| C complex | CACTIN | C complex |
| C complex | NOSIP | C complex |
| C complex | WDR83 | C complex |
| C complex | FAM50A | C complex |
| C complex | PPIG | C complex |
| C complex | SDE2 | C complex |
| C complex | CDK10 | C complex |
| C complex | LENG1 | C complex |
| C complex | FAM32A | C complex |
| C complex | FRA10AC1 | C complex |
| C complex | PRPF18 | second step factor |
| C complex | SLU7 | second step factor |
| C complex | DHX8 | second step factor |
| C complex | MAGOH | EJC |
| C complex | RBM8A | EJC |
| C complex | HNRNPC | hnRNP |
| C complex | SRRM2 | SR related |
| | SART3 | U4/U6 recycling |
| | DHX38 | second step factor |
| | LUC7L | U1 snRNP |
| | DDX42 | 17S U2 snRNP associated |
| | PRPF4B | B complex |
| | YJU2 | Bact complex |
| | RNPS1 | EJC |
| | ALYREF | EJC |
| | NXT1 | EJC |
| | NXF1 | EJC |
| | CASC3 | EJC |
| | ACIN1 | EJC |
| | UPF1 | EJC |
| | PNN | EJC |
| | PRPF38B | |
| | TCERG1 | |
| | SKIV2L2 | |
| | SNRNP27 | |
| | RUVBL1 | |
| | PRPF39 | |
| | MOV10 | |
| | GPATCH11 | |
| | C16orf80 | |
| | SRPK1 | |
| | SRPK2 | |
| | DBR1 | |
| | HNRNPUL1 | hnRNP |
| | FUS | hnRNP |
| | HNRNPAO | hnRNP |
| | PCBP1 | hnRNP |
| | PCBP2 | hnRNP |
| | HNRNPA2B1 | hnRNP |
| | HNRNPA3 | hnRNP |
| | HNRNPAB | hnRNP |
| | HNRNPD | hnRNP |
| | HNRNPF | hnRNP |
| | RBMX | hnRNP |
| | HNRNPH1 | hnRNP |
| | HNRNPH3 | hnRNP |
| | HNRNPK | hnRNP |
| | HNRNPL | hnRNP |
| | HNRNPM | hnRNP |
| | HNRNPR | hnRNP |
| | HNRNPU | hnRNP |
| | RALY | hnRNP |
| | SYNCRIP | hnRNP |
| | HNRNPH2 | hnRNP |
| | HNRNPUL2 | hnRNP |
| | HNRNPDL | hnRNP |
| | RBMXL2 | hnRNP |
| | HNRNPCL1 | hnRNP |
| | SRSF1 | SR protein |
| | SRSF2 | SR protein |
| | SRSF4 | SR protein |
| | SRSF5 | SR protein |
| | SRSF6 | SR protein |
| | SRSF7 | SR protein |
| | SRSF11 | SR protein |
| | SRSF9 | SR protein |
| | SREK1 | SR protein |
| | TRA2B | SR protein |
| | TRA2A | SR protein |
| | SRSF3 | SR protein |
| | SFSWAP | SR protein |
| | SRSF12 | SR protein |
| | SRSF8 | SR protein |
| | SRSF10 | SR protein |
| | SRRM1 | SR related |

As used herein, the term “target” refers to a component of RNA metabolism that is desired to be inhibited. In specific embodiments, the target is an RNA helicase or splicing regulator that is desired to be inhibited specifically by one or more inhibitors. Any RNA helicase target may or may not be directly involved in splicing. In specific cases, the target is bound directly by the one or more inhibitors to effect the inhibition.

As used herein, the term “test inhibitor” or “candidate inhibitor” refers to a molecule that is being tested by one or more particular assays to identify targeting of a desired protein and also to identify absence of targeting of proteins not desired to be targeted by the inhibitor.

The present disclosure contemplates systems and methods that produce selective inhibitors of RNA metabolism, including at least RNA splicing; in specific cases, the selective inhibitors target one or more RNA helicases or splicing regulator but do not target other one or more RNA helicases or splicing regulator. The systems and methods utilize chemical, genetic, and/or computational means to characterize a plurality of RNA helicases to the extent that drug testing (through analysis of candidate inhibitors) identifies suitable inhibitors that selectively inhibit desired RNA helicases having common structure (for example) but are counter-selective for inhibiting RNA helicases that are not desired and, in at least some cases, may result in toxicity for a cell, tissue, or organism.

In particular embodiments, the multi-faceted approach of the disclosed subject matter allows for systematically targeting RNA helicases or splicing regulators to produce inhibitors that are drugs to target splicing in cancer, autoimmune disease, infectious disease, and neurodegeneration, including mis-splicing directly or indirectly related to cancer, autoimmune disease, infectious disease, and neurodegeneration. These selective inhibitors against certain RNA helicases, but not other RNA helicases, allow for inhibition of RNA helicases that are master regulators of RNA splicing in cancer, autoimmune disease, infectious disease, and neurodegeneration, in at least some cases.

The selectivity aspects of the systems and methods allow for structural and functional differentiation of related RNA helicases to assist in identifying which inhibitors may be applicable to a particular group of structurally and functionally related RNA helicases. The produced signatures from the systems and methods allow for probing in-cell selectivity of RNA helicase inhibitors. The disclosure demonstrates differentiation of structurally and functionally related RNA helicases, including by producing signatures for probing in-cell selectivity of RNA helicase inhibitors.

In specific embodiments of the disclosure, processes utilized in-cell or in vivo RNA helicase degradation-based systems that produce and define a RNA signature that represents specific inhibition of the helicase; this is then followed by subjecting one or more candidate inhibitors to cells and determining if the same or similar RNA signature is produced. Such inhibitor screening methods are configured to determine if one or more particular candidate inhibitors are selective.

I. Cellular Systems and Uses Thereof

Embodiments of the present disclosure encompass in-cell, in-tissue, and/or in vivo analysis of RNA helicases using cellular systems. Such analysis identifies suitable RNA helicase(s) for selective targeting, and then identification of suitable inhibitors that selectively target those RNA helicase(s).

In specific embodiments, RNA helicases are characterized to determine their role in specific cancer dependencies or those related to autoimmune disease, infectious disease, and neurodegeneration. At least part of this characterization includes development of in-cell and in-tissue assays for selectivity and pharmacodynamic biomarker identification. These methods utilize a cellular platform comprising cancer cell lines, normal cell lines, and/or animal models in which at least one RNA helicase target or splicing regulator target is disrupted such that a functional RNA helicase or splicing regulator respectively is not produced for one or a variety of reasons. The disruption may be disruption at the gene level or at the mRNA or protein level. In specific cases, the disruption is from being selectivity degraded, such as by an enzymatic mechanism. The information from these actions is applicable to identification of suitable inhibitors of the disrupted RNA helicase(s) or splicing regulator(s). The platform enables modeling of selective RNA helicase inhibition prior to production of tool or clinical-grade compounds and that will provide informative guidance to such production.

In specific cases, use of the disclosed cellular systems will model inhibition/degradation of individual RNA helicases across a diverse family of RNA helicases to provide information of shared and selective functions (MOA) for targeting RNA helicase(s) of interest. Following this inhibition, the systems permit identification of selectivity signatures and assays for assessing in-cell activity of candidate inhibitors against the target RNA helicase(s) of interest. In some embodiments, RNA helicases being disrupted in methods of the disclosure and that are involved in splicing may be characterized in assays other than those that test splicing, for example when a role in splicing is unknown or to identify helicases having multiple roles in biological processes. In other cases, helicases being disrupted in methods of the disclosure and that are not known to be involved in splicing may be characterized in assays that test splicing.

In particular embodiments, output from the in-cell assays or in-tissue assays or in vivo assays comprises information about which RNA helicases, whether or not used for RNA splicing, when inhibited affect the same or similar gene product pattern. This information may be utilized for identification of pharmacodynamic (PD) biomarkers for targeting one or more RNA helicases of interest. In particular, the output of one or more assays encompassed herein identifies the PD biomarkers as molecular indicators of the effect of the candidate inhibitors on the target RNA helicase in a cell, tissue, or organism. A PD biomarker may be used to characterize the association between drug regimen, target RNA helicase effect, and at least in some cases, response of cancer cells in vivo. Such characterization provides suitable data to make informed decisions to select rational combinations of targeted agents and/or to optimize regimens of drug therapy. The PD biomarkers enhances decisions throughout drug development, including selection of lead compounds in preclinical models to first-in-human trials.

In particular embodiments, the assays identify predictive biomarkers for patient selection for inhibitors against the target RNA helicase(s) of interest. In at least some cases, methods encompassed herein in which an RNA helicase (for example) is disrupted, resulting in a particular RNA signature, provides information that is applied in a clinical setting. For example, when an individual with a medical condition (whether or not it is cancer, autoimmune disease, neurodegeneration, or infectious disease) has samples having the same or similar RNA signature that is produced when a particular RNA helicase is disrupted, then one or more inhibitors for that particular RNA helicase may then be provided to the individual for treatment of the medical condition.

Thus, in particular embodiments the cellular embodiments of the system and methods allows the following: (1) target validation, prioritization, and biology—characterize shared and selective functions of target RNA helicases of interest, (2) enablement of lead identification and lead optimization by providing signatures and assays for quantifying in-cell activity and selectivity of small molecule inhibitors against target RNA helicase of interest, (3) enablement of identification of pharmacodynamics markers/assays by providing signatures and assays for quantifying in-tissue activity and selectivity of small molecule inhibitors against target RNA helicase of interest, (4) enablement of identification of predictive biomarkers for patient selection (“precision medicine”).

Specific aspects of the disclosure allow for identification of MOA of targets and delineation of biomarkers, both pharmacodynamic (PD) biomarkers and/or predictive markers, and the associated assays that come from the discovery of those markers. The information from this identification facilitates development of one or a set of assays that specifically measure those markers. The disclosure provides in-cell assays for selectivity during drug discovery and assays in pre-clinical models to delineate PD markers linked to a perturbation (e.g., inhibition of RNA helicase(s) by any means, including by degradation), and this may include one or more assays in patients as PD markers for medicines.

Particular embodiments of the disclosure analyze an RNA helicase family and, in specific aspects, more broadly for splicing (for example, regulators of splicing). In particular embodiments, each member of the family is analyzed by inhibiting their function by chemical-genetic means. In specific cases, the inhibition of their function occurs by perturbation of individual RNA helicases or splicing regulators, and this can be accomplished by a variety of perturbation means, including at least enzymatic, such as marking by any means the RNA helicase or splicing regulator for destruction by the proteosome. Following perturbation of each of the plurality of RNA helicases, one can make large-scale measurements of RNA splicing, such as through one or more various forms of sequencing, including next generation sequencing (NGS). In specific embodiments, the NGS includes part or all of an intron and/or may include sequencing of particular exon/intron junctions. When a pre-mRNA has multiple introns, one or more of the introns and/or their junctions may or may not be sequenced. In specific cases, a particular one or more introns for a particular one or more genes is selected for sequencing and, in certain aspects, the selection involves known points of mis-splicing by RNA helicases associated with one or more particular cancers, autoimmune disease, infectious disease, and neurodegeneration. Methods for analysis of RNA splicing are known in the art and may utilize NGS, Q-PCR, Nanostring, HiBit, etc.

In a specific case, there is generation of parallelized data sets to allow contrast and comparison among particular inhibited RNA helicases. For example, when a first particular RNA helicase is disrupted, such as at the gene or protein level, a particular resultant pattern of RNA metabolism parameters (mRNA processing, double stranded RNA accumulation, differential expression, differential splicing, or a combination thereof) is obtained. Then, when a second or subsequent particular RNA helicase is disrupted, such as at the gene or protein level, a second or subsequent particular resultant pattern of RNA metabolism parameters (mRNA processing, double stranded RNA accumulation, differential expression, differential splicing, or a combination thereof) is obtained. These patterns may be compared and contrasted, and the information obtained from this may be applied to downstream applications, such as grouping of a plurality of helicases that may be able to be inhibited by a common inhibitor, or grouping of a plurality of helicases that should not be inhibited because doing so would be toxic to a cell or organism. In a specific example of the generation of parallelized data sets as just described, the comparisons/contrasts among a plurality of RNA helicases facilitates determination of a set of RNAs/introns that are specifically sensitive to inhibiting one or more RNA helicase of a first subset of the plurality but not inhibiting one or more RNA helicases of a second subset of the plurality. Thus, the comparisons/contrasts may comprise information about the transcriptome effect of inhibiting a first subset of the RNA helicases from the plurality compared to inhibiting a second or subsequent subset of RNA helicases from the plurality. In a particular embodiment, a particular subset of the RNA helicases may comprise, or consist of, or consist essentially of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 RNA helicases of the following list of 73 helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27. In specific cases, the generation of parallelized data sets and/or analyses of comparisons and contrasting may be performed by a computer, including using an algorithm.

In certain embodiments, markers produced by analyses encompassed herein provide information for when the inhibitors would be suitable as a drug for a patient, including how one can measure the effect of the drug. As an example, the information facilitates generation of assays useful for testing drugs in a patient, including what measurement(s) can be made to ascertain when dose escalation may be warranted, for example. In some embodiments, the systems and methods measure information for multiple RNA helicase targets with clearly defined patient indications, such as being associated with specific RNA mis-splicing associated with a medical condition, such as cancer, autoimmune disease, infectious disease, or neurodegeneration. The systems and methods of the disclosure also identify genotype-selective vulnerabilities in RNA mis-splicing. For example, an individual having cells with a specific RNA signature pattern that is similar to or the same as RNA signature patterns associated with RNA mis-splicing (whether natural or produced upon selective inhibition of one or more RNA helicases) may have or be susceptible to one or more medical conditions linked to RNA mis-splicing, such as cancer, autoimmune disease, neurodegeneration, infectious disease, and so forth.

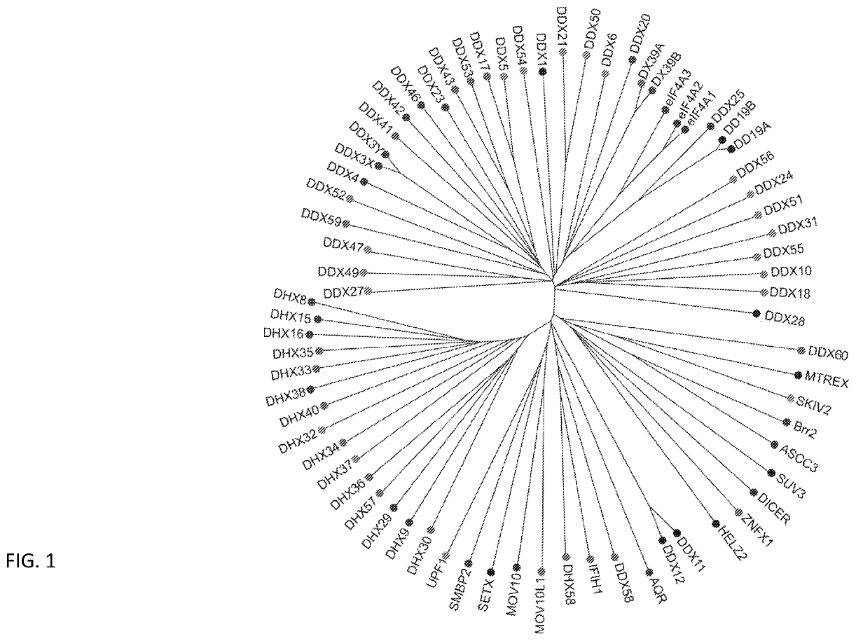

In particular embodiments, a plurality of RNA helicases characterized by systems and methods herein are of a particular group. FIG. 1 illustrates a phylogenetic tree demonstrating the evolutionary relationships among certain RNA helicases and based upon sequence similarity, and in specific embodiments the systems and methods of the disclosure employ parallelized helicase degradation as a means for determining which inhibitors are useful to inhibit one or more desired helicases yet avoid inhibition of essential RNA helicases that could result in toxicity in a cell or in vivo.

The following includes a list of members of the phylogenic tree of FIG. 1: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27. Any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 or all 73 of the proteins listed in FIG. 1 may be an RNA helicase that is a target for screening for an inhibitor. In some cases, one or more of these are not to be targeted because target perturbation studies provide information determining that their inhibition would be toxic to a cell, tissue, or organism.

In particular embodiments for the plurality of RNA helicases listed in FIG. 1, cancer or other diseased cell types are more sensitive to RNA helicase inhibition than normal cells, and these may be the RNA helicases that are targeted. In other cases, there is a subset of the plurality that is suitable for targeting because they are non-essential (not toxic when inhibited) and there is another subset of the plurality that is not suitable for targeting because those in the subset are essential (toxic when inhibited). The plurality of multiple RNA helicases that are suitable for targeting may or may not be in the same subset because they are structurally related, such as having a certain percentage identity between two RNA helicases or having a certain percentage identity among more than two RNA helicases. The plurality of multiple RNA helicases that are suitable for targeting may or may not be in the same subset because they are functionally related.

Methods of the disclosure also encompass tests in a patient or research sample wherein a known RNA signature from disruption of one or more RNA helicases or splicing regulators is utilized to determine whether or not a drug unknown to be an inhibitor of a helicase actually inhibits the helicase, such as an unknown off-target property of the drug. In specific cases, the drug in question has a toxicity because of an unknown off-target inhibition of a particular RNA helicase or splicing regulator based on the signature. If an inhibitor has a toxicity and it may inhibit a particular RNA helicase, one can perform studies to determine if use of the inhibitor against a cell produces an RNA signature that is the same or substantially the same as an RNA signature pattern for a certain RNA helicase known to be associated with toxicity upon its inhibition. In such a case, one can predict that the inhibitor likely targets the particular helicase and that such targeting of the particular helicase may lead to the same or similar toxicity.

In specific cases, one may have a drug of any kind and have a desire to determine if it can produce a toxicity, including based on inhibition of one or more RNA helicases and/or one or more splicing regulators. In specific cases, the drug is unknown whether it has such inhibition but upon delivery of the drug to cells, an RNA signature is produced that is the same or substantially similar to the RNA signature pattern produced upon inhibition of one or more RNA helicases and/or one or more splicing regulators that is toxic to cells. In such cases, the drug may be predicted to be toxic to a cell and/or an individual. Thus, methods of the disclosure encompass methods of predicting toxicities to a drug.

In some cases, an inhibitor may inhibit more than one helicase and/or splicing regulator. In such cases, testing of the inhibitor may produce a first RNA signature pattern upon inhibition of a first helicase or splicing regulator, and it may produce a second RNA signature pattern (non-identical to the first RNA signature pattern) upon inhibition of a second helicase or splicing regulator. There may be overlap of RNA signatures produced upon inhibition of two different helicases or splicing regulators by the same inhibitor.

II. Computational Systems

Computational systems as encompassed herein facilitate analysis of data from one or more screens or assays of any kind that concern analysis of target protein function and/or testing of candidate compounds for those target proteins. In particular embodiments, computational systems are utilized to recognize or identify patterns of information produced from certain methods encompassed herein, including with respect to target protein function, candidate compounds that may activate or inhibit one or more target proteins, RNA signatures produced upon exposure of cells to one or more compounds, or a combination thereof. In specific cases, the disclosure provides one or a new set of algorithms that facilitate determination of patterns of RNA signatures produced upon normal or aberrant splicing and, in some cases, with respect to processing of one or more RNA helicases or splicing regulators, including through inhibition, for example.

In particular embodiments, utilization of the computational system, which in specific cases is used in conjunction with cellular processes, provides information when screening and characterizing drug development. Some information concerns whether one or more test inhibitors inhibit only a desired helicase or splicing regulator or whether one or more test inhibitors also inhibit one or more helicases related to a desired helicase or splicing regulators related to a desired splicing regulator, respectively. Also, the computational information allows for recognition of patterns in RNA metabolism, such as with respect to the transcriptome, for inhibition of, e.g., a first RNA helicase and also a closely related one or more RNA helicases. Such information may be used directly or indirectly to design or perform assays used to drive in-cell or in vivo selectively of candidate drugs.

In particular embodiments, computational systems are employed to facilitate analysis of information in which one or more RNA signatures are compared to one or more other RNA signatures. In some cases, one or more RNA signatures produced upon testing conditions are compared to one or more known RNA signatures. In some cases, whether or not the one or more RNA signatures produced upon testing conditions are the same or substantially the same may be informative, such as determining whether or not a particular test compound (inhibitor or activator) would be suitable for a specific purpose. In cases wherein the one or more RNA signatures produced upon testing conditions are not the same or substantially the same may also be informative, also for determining whether or not a particular test compound (inhibitor or activator) would be suitable for a specific purpose. In one example, a computational system is utilized that recognizes a pattern for when a particular test compound is the same or substantially the same as the pattern of one or more known RNA signatures produced upon inhibition of an RNA helicase(s) (or splicing regulator(s)) that are essential for cellular processes. In such a case, the comparison produced by the computational analysis would indicate that the particular test compound should not be utilized. As used herein, an RNA signature is substantially the same as another RNA signature if in their respective collection they share the majority of specific RNA molecules having particular splicing events or patterns with respect to individual RNA molecules. In a specific case, an RNA signature is substantially the same as another RNA signature if they share greater than 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% of specific RNA molecules having particular splicing events or patterns with respect to individual RNA molecules.

In particular embodiments, the computational systems and methods characterize RNA helicase MOA and characterize how these mechanisms lead to specific cancer dependencies or those related to autoimmune disease, infectious disease, and neurodegeneration. The computational system and methods also encompass or allow for development of in-cell and in-tissue assays for selectivity and identification of pharmacodynamics markers. In a specific embodiment, the computational platform encompassed herein comprises proprietary algorithm(s) and methods to quantify global and event-level changes in RNA processing. This platform enables identification of pharmacodynamic markers and signatures with respect to inhibition of one or more specific RNA helicases and in some cases such information can be utilized to quantify in-cell and in-tissue activity and selectivity of RNA helicase small molecule inhibitors.

In certain embodiments, computational systems facilitate analysis of compounds suitable for identifying activators or inhibitors of immune signaling. As one example, a computer identifies an RNA signature having accumulation of particular misprocessed RNAs that themselves activate antiviral immune signaling and/or antitumor immune signaling. Computer analysis may include comparisons of RNA signatures comprising specific misprocessed RNAs that activate antiviral immune signaling and/or antitumor immune signaling. In a specific case, an RNA signature produced following subjecting of a candidate activator to certain cells is compared by computer analysis to RNA signatures produced from one or a plurality of disrupted RNA helicases and/or splicing regulators (and these may be known). The computer analysis may include pattern recognition such that it detects whether or not an RNA signature produced by a candidate activator is the same or substantially the same as RNA signatures produced from one or a plurality of disrupted RNA helicases and/or splicing regulators, and this information determines when a candidate activator is an activator or not. Analogously, computer analysis may be utilized to identify patterns related to RNA signatures produced by candidate inhibitors of antiviral immune signaling and/or antitumor immune signaling.

III. Inhibitors

The systems and methods of the disclosure include in-cell or in vivo screens for identifying inhibitors of RNA helicases or splicing regulators, including for clinical purposes. The inhibitors being assayed in the disclosed systems and methods are candidate inhibitors while determination is made as to their suitability for selective inhibition of RNA helicases or splicing regulators. A candidate inhibitor that selectively inhibits desired one or more RNA helicases or splicing regulators but that does not inhibit other RNA helicases or splicing regulators that are essential is an inhibitor that may be applied for clinical applications.

The inhibitors may be obtained from any source, including generated de novo or obtained from a library, whether commercial or not. The inhibitors may be selected for having one or more structural or functional attributes, such as similarity to other known RNA helicase inhibitors or based on structure-activity relationship analysis. In other cases, one or more attributes for the inhibitors include a structural component that is known or suspected of being useful to inhibit functionality of certain enzymatic mechanisms of action. In other cases, the inhibitors are selected from a library without any knowledge of useful attributes for the inhibitors.

In some embodiments, the inhibitor may be any type of inhibitor and any type of molecule. Although in particular embodiments the inhibitor is a small molecule, in alternative embodiments the inhibitor is not a small molecule, such as a protein, peptide, nucleic acid, carbohydrate, or a combination thereof. The term “small molecule” as used herein refers to an organic compound having a size of less than 1500 Daltons.

These compounds may be competitive with the NTP site, competitive with the RNA binding site, competitive with an accessory protein binding site, bind to an allosteric site to block access to one or both of the NTP and RNA binding sites, enhance binding to preclude hydrolysis of the NTP, enhance binding but restrict the processivity of across the RNA species, or have another allosteric function.

IV. Methods of Treatment

In particular embodiments, the methods encompassed herein identify one or more compounds that are useful for treatment of any medical condition. In some cases, the medical condition is directly or indirectly treated based on administration of one or more compounds identified herein. In some cases, the medical condition is treated by a compound that directly or indirectly impacts RNA metabolism, including RNA processing of any kind. In specific cases, the medical condition is treated by a compound that targets an RNA helicase or splicing regulator, either through inhibition or activation. That is, in some cases a compound identified by methods herein inhibits a defective RNA helicase that without the inhibition produces an accumulation of misprocessed RNA associated with cancer. In some cases a compound identified by methods herein inhibits an RNA helicase that then increases accumulation of certain misprocessed RNA that activates antitumor signaling and/or antiviral signaling. In any event, a therapeutically effective amount of one or more inhibitors screened from or otherwise identified by methods disclosed herein may be provided to an individual in need thereof. In some cases, the medical condition includes cancer of any kind, autoimmune disease, infectious disease, neurodegeneration, and so forth.

Any cancers disclosed herein may have defective splicing for which the inhibitors identified by methods encompassed herein are therapeutic for the cancer. In some embodiments, an RNA helicase is targeted as being associated with aberrant splicing because the RNA helicase is mutated, and the mutation may be the direct or indirect cause of the medical condition. Regardless of action, in some embodiments, the inhibitors identified through screens herein are useful for alleviation of at least one symptom in cancer, in specific cases. The cancer may be of any type or grade or tissue of origin. It may or may not be metastatic. Tumors for which the inhibitors identified through methods disclosed herein are useful include any malignant cell type, such as those found in a solid tumor or a hematological tumor. In some cases, the inhibitor targets one or more defective RNA helicases that are associated with chemo-refractory malignancies.

Specific cancers for which the inhibitors identified through methods disclosed herein are useful include non-small cell lung cancer adenocarcinoma, ovarian cancer, esophageal cancer, HCC, head and neck cancer, non-small cell lung squamous cancer, breast cancer (including at least triple-negative), gastric cancer, pancreatic cancer, bladder cancer, colon cancer, cecum cancer, stomach cancer, brain cancer, kidney cancer, larynx cancer, sarcoma, lung cancer, melanoma, prostate cancer, and so on. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In certain embodiments, compounds identified by screening or other methods encompassed herein are provided in a therapeutically effective amount to an individual with an autoimmune disease. In specific cases, the autoimmune disease is the result directly or indirectly of aberrant splicing. In particular embodiments, the autoimmune disease is selected from the group consisting of Type 1 diabetes, rheumatoid arthritis, psoriasis, multiple sclerosis, Systemic lupus erythematosus, Graves' disease, inflammatory bowel disease, Addison's disease, Sjögren's syndrome, Hashimoto's thyroiditis, Myasthenia gravis, celiac disease, Autoimmune vasculitis, Pernicious anemia, Dermatomyositis, and so forth.

In certain embodiments, the compounds identified by screening or other methods encompassed herein are provided in a therapeutically effective amount to an individual with neurodegeneration, such as associated with neurodegenerative diseases including at least amyotrophic lateral sclerosis, multiple sclerosis, Parkinson's disease, Alzheimer's disease, Huntington's disease, and prion diseases.

In some embodiments, the compounds identified by screening or other methods encompassed herein are provided in a therapeutically effective amount to an individual with an infectious disease. The infectious disease may be of any kind, including at least bacterial, viral, fungal, or parasitic.

Examples of viruses associated with infectious disease include, but are not limited to, at least adenovirus, alphavirus, calicivirus, coronavirus (including SARS CoV2 and SARS CoV), distemper virus, Ebola virus, enterovirus, flavivirus, hepatitis virus, herpesvirus (including herpes simplex virus or varicella zoster virus), infectious peritonitis virus, influenza virus, leukemia virus, Marburg virus, orthomyxovirus, papilloma virus, parainfluenza virus, paramyxovirus, parvovirus, pestivirus, picorna virus, pox virus, rabies virus, reovirus, retrovirus, and rotavirus. Specific viruses include at least human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, and so forth.

Examples of bacteria associated with infectious disease include, but are not limited to, at least Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea, Salmonella, Shigella, Staphylococcus, Streptococcus, Treponema, and Yersinia.

Examples of fungus associated with infectious disease include, but are not limited to, at least Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha.

Examples of protozoa associated with infectious disease include, but are not limited to, at least Acanthocheilonema, Aelurostrongylus, Ancylostoma, Angiostrongylus, Ascaris, Babesia, Balantidium, Besnoitia, Brugia, Bunostomum, Capillaria, Chabertia, Cooperia, Crenosoma, Cryptosporidium, Dictyocaulus, Dioctophyme, Dipetalonema, Diphyllobothrium, Diplydium, Dirofilaria, Dracunculus, Enterobius, Eimeria, Encephalitozoon, Entamoeba, Filaroides, Giardia, Haemonchus, Hammondia, Hepatozoon, Isospora, Lagochilascaris, Leishmania, Loa, Mansonella, Microsporidia, Muellerius, Nanophyetus, Necator, Nematodirus, Neospora, Nosema, Oesophagostomum, Onchocerca, Opisthorchis, Ostertagia, Parafilaria, Paragonimus, Parascaris, Pentatrichomonas, Physaloptera, Plasmodium, Pneumocystis, Protostrongylus, Sarcocystis, Schistosoma, Setaria, Spirocerca, Spirometra, Stephanofilaria, Strongyloides, Strongylus, Theileria, Thelazia, Toxascaris, Toxocara, Toxoplasma, Trichinella, Trichostrongylus, Trichuris, Trypanosoma, Uncinaria, and Wuchereria.

Compounds identified from methods encompassed herein may be formulated specifically for therapeutic use. In specific cases the inhibitor identified by screening methods and symptoms herein is formulated in a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In specific embodiments, the disclosure encompasses methods of treating a subject (including a mammal such as a human) having a medical condition associated with defective RNA metabolism comprising administering to the subject a pharmaceutical composition comprising a therapeutically effective amount of a selective inhibitor of one or more RNA helicases identified by any method herein.

In one embodiment, there is a method of treating a subject having a medical condition (such as cancer, including solid tumor or hematological) associated with defective RNA metabolism comprising administering to the subject a pharmaceutical composition comprising a therapeutically effective amount of a selective inhibitor of one or more RNA helicases, wherein the inhibitor is identified by an in vivo screening method comprising the steps of providing an in-cell or in vivo assay for disruption of one or more RNA helicases or one or more splicing regulators; measuring one or more RNA metabolism parameters (e.g., RNA splicing, RNA decay, RNA catabolism, RNA export, transcription, translation, rRNA biogenesis, RNA modification, or a combination thereof) including RNA transcripts and/or proteomics in the cell following said disruption; and subjecting one or more test candidates to the in-cell or in vivo assay for each RNA helicase in a plurality of helicases for identification of test candidates that modify the one or more RNA metabolism parameters and/or proteomics for a first subset of RNA helicases and/or splicing regulators in the plurality but that do not modify the one or more RNA metabolism parameters and/or splicing regulators for a second subset of RNA helicases in the plurality. In specific embodiments, the defective RNA metabolism is defective synthesis, folding/unfolding, modification, processing, stabilization, or degradation of RNA; accumulation of misprocessed RNA; and/or defective RNA splicing. The disruption may be at the gene level or at the mRNA or protein level. The disruption may be carried out by enzymatic degradation (including degradation by the proteasome or phagosome), small molecule inhibition of enzymatic activity, allosteric small molecule inhibitor, or small molecule degrader. In some embodiments, the disruption is at the gene level or at the mRNA level. The disruption may be carried out by RNAi, siRNA, shRNA, ribozymes, CRISPRCas9, homologous recombination, site-specific nucleases, zinc fingers, or TALENs. The cells of the in-cell assay may be normal or not cancerous, or cancerous, such as cancerous cells are breast cancer, non-small cell lung cancer, small cell lung cancer, colorectal cancer, all subtypes of leukemia, all subtypes of lymphoma, ovarian cancer, esophageal cancer, hepatic cell carcinoma, head and neck cancer, gastric cancer, pancreatic cancer, or bladder cancer. The method may further comprise the step of formulating said inhibitors in a pharmaceutically acceptable carrier

With respect to the measuring step, it may comprise measuring double stranded RNA, mRNA processing, differential expression, differential splicing, global RNA processing fidelity, RNA catabolites, protein levels, RNA substrates, RNA binding motifs, or a combination thereof. The measuring step may comprise measuring intron splicing in one or more genes upon degradation of one or more RNA helicases or measuring intron splicing in multiple genes upon degradation of multiple RNA helicases. Information from measuring intron splicing in multiple genes upon degradation of multiple RNA helicases may result in identification of inhibitors that modify RNA splicing for a first subset of RNA helicases in the plurality but that do not modify RNA splicing for a second subset of RNA helicases in the plurality.

For the subjecting step, in some embodiments the subjecting step is further defined as subjecting one or more test candidates to the in-cell assay for each RNA helicase in the plurality to identify test candidates that modify the one or more parameters of RNA metabolism or proteomics for a first subset of RNA helicases in the plurality, followed by identifying the absence of modifying the one or more parameters of RNA metabolism or proteomics for the second subset of RNA helicases in the plurality. The subjecting step, which may comprise high throughput screening, may be further defined as subjecting one or more test candidates to the in-cell assay for each RNA helicase in the plurality to identify test candidates that do not modify the one or more parameters of RNA metabolism or proteomics for the second subset of RNA helicases in the plurality, followed by identifying the presence of modifying for the one or more parameters of RNA metabolism or proteomics for the first subset of RNA helicases in the plurality. The subjecting step may be further defined as subjecting one or more test candidates to the in-cell assay for each RNA helicase in the plurality to identify test candidates that modify the one or more parameters of RNA metabolism or proteomics for a first subset of RNA helicases in the plurality at substantially the same time as identifying the absence of modifying the one or more parameters of RNA metabolism or proteomics for the second subset of RNA helicases in the plurality.

In some embodiments, the test candidates are small molecules, proteins, peptides, nucleic acid, carbohydrate, or a combination thereof.

RNA helicases in the first subset of RNA helicases may be from the same sub-family of RNA helicases. RNA helicases in the first subset of RNA helicases may share the same function in RNA metabolism, which may be RNA splicing. The first subset of RNA helicases may comprise one or more RNA helicases, and the second subset of RNA helicases may comprise one or more RNA helicases. In some cases, the RNA splicing by the RNA helicase(s) in the first subset of RNA helicases is aberrant in cancer cells. The plurality of RNA helicases comprises two or more, the majority of, or all of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

In certain embodiments of the method, identification of test candidates that modify the one or more RNA metabolism parameters for a first subset of RNA helicases in the plurality but that do not modify the one or more RNA metabolism parameters for a second subset of RNA helicases in the plurality is performed by a computer. Identification of test candidates that modify the one or more RNA metabolism parameters for a first subset of RNA helicases in the plurality but that do not modify the one or more RNA metabolism parameters for a second subset of RNA helicases in the plurality may utilize an algorithm.

In some embodiments, the medical condition is cancer, and the inhibitor may target one or more defective RNA helicases that are associated with chemo-refractory malignancies. The cancer may be selected from the group consisting of non-small cell lung cancer adenocarcinoma, ovarian cancer, esophageal cancer, HCC, head and neck cancer, non-small cell lung squamous cancer, breast cancer (including at least triple-negative), gastric cancer, pancreatic cancer, bladder cancer, colon cancer, cecum cancer, stomach cancer, brain cancer, kidney cancer, larynx cancer, sarcoma, lung cancer, melanoma, prostate cancer, tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma. The cancer may be a histological type comprising neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In some embodiments, the medical condition being treated is an autoimmune disease, including one that is a result of direct or indirect aberrant splicing. In specific embodiments, the autoimmune disease is selected from the group consisting of Type 1 diabetes, rheumatoid arthritis, psoriasis, multiple sclerosis, Systemic lupus erythematosus, Graves' disease, inflammatory bowel disease, Addison's disease, Sjögren's syndrome, Hashimoto's thyroiditis, Myasthenia gravis, celiac disease, Autoimmune vasculitis, Pernicious anemia, and Dermatomyositis.

In some embodiments, the medical condition being treated is a neurodegenerative disease, such as one selected from the group consisting of amyotrophic lateral sclerosis, multiple sclerosis, Parkinson's disease, Alzheimer's disease, Huntington's disease, and prion diseases.

In some embodiments, the medical condition is an infectious disease, including an infectious disease that is at least bacterial, viral, fungal, or parasitic. The infectious disease may be selected from the group consisting of adenovirus, alphavirus, calicivirus, coronavirus (including SARS CoV2 and SARS CoV), distemper virus, Ebola virus, enterovirus, flavivirus, hepatitis virus, herpesvirus (including herpes simplex virus or varicella zoster virus), infectious peritonitis virus, influenza virus, leukemia virus, Marburg virus, orthomyxovirus, papilloma virus, parainfluenza virus, paramyxovirus, parvovirus, pestivirus, picorna virus, pox virus, rabies virus, reovirus, retrovirus, and rotavirus. Specific viruses include at least human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), and adenovirus.

The pharmaceutical composition utilized in the method may further comprise a pharmaceutically acceptable carrier, including one that is selected from the group consisting of solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and any combinations thereof. The pharmaceutically composition may be administered in solid, liquid or aerosol form. The pharmaceutically composition may need to be sterile for administration as injection. The pharmaceutically composition may be administered by any suitable method, including intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by any combinations thereof. The subject being treated may be a human.

V. Activators or Inhibitors of Antiviral Immune Signaling and/or Antitumor Immune Signaling

Inhibition of splicing can cause accumulation of misprocessed RNAs and folding of RNA that is analogous to virus structures and can cause this misprocessed endogenous RNA to stimulate an antiviral response in vivo (in cells). This phenomena is more pronounced in tumor cells and other disease states. In tumors, analogously the misprocessing of RNAs has an effect in tumor cells, leading to activation of an antitumor immune response in the body.

Inhibition of RNA helicases associated with splicing also produces accumulation of misprocessed RNAs, and this can mimic the natural activation of antiviral immune pathways and/or antitumor immune pathways. The present methods of the disclosure may be utilized to measure how candidate compounds can affect antiviral and/or antitumor immunity. In at least specific cases, the methods of the disclosure allow production of output(s) related to stimulation of antiviral and/or antitumor immunity.

In particular embodiments, the disclosure concerns identification of compounds for manipulation of antiviral immune pathways and antitumor immune pathways in-cell or in vivo. Such compounds in specific cases are useful as an immuno-oncological treatment because they exploit the body's immune system to fight cancer by mimicking modulators of antiviral immune pathways and antitumor immune pathways.

Embodiments of the disclosure provide assays to identify activators or inhibitors of the immune system (including antiviral or antitumor processes) based on information procured from disruption of one or more RNA helicases or splicing regulators, as in methods disclosed herein. In specific cases, the RNA signatures obtained from disruption of specific RNA helicases and/or splicing regulators is applied to analysis of compounds that can be tested for modulating the immune system. In specific embodiments, certain misprocessed RNAs following disruption of particular RNA helicases and/or splicing regulators, but not others, are those that activate the antiviral immune signaling and/or the antitumor immune signaling. The disclosed methods allow one to identify patterns that connect disruptions of RNA helicases and/or splicing regulators with modulation of the immune system. A misprocessed RNA may be determined to be one that activates antiviral immune signaling and/or antitumor immune signaling by standard methods in the art, such as by identifying repetitive elements in the misprocessed RNA, including partially or fully within the intron. In additional or other cases, one may determine whether the misprocessed RNA binds one or more proteins that recognize a virus and/or that is linked to signaling in antiviral immune signaling and/or antitumor immune signaling.

In certain cases, it is desirable to manipulate antiviral immune signaling and/or antitumor immune signaling in-cell or in vivo by stimulating these pathways that would directly or indirectly result in recruitment or activation of at least anti-tumor immunity. To do so, one would enhance the presence of misprocessed RNAs that stimulate the antiviral immune system and/or the antitumor immune system in-cell or in vivo. To be able to enhance the presence of such misprocessed RNAs, one would disrupt RNA helicase(s) and/or splicing regulator(s) that result in accumulation of misprocessed RNAs that are capable of such stimulation. Therefore, a compound that inhibits particular RNA helicase(s) and/or splicing regulator(s) would be desired if one were to activate antiviral immune signaling and/or antitumor immune signaling. One example of an application for such a compound would be to use it to treat cancer or infectious disease.

In alternative cases, it is desirable to inhibit innate immune (antiviral) pathways in-cell or in vivo that are abnormally activated by the presence of aberrant RNA splicing. Such abnormal activation may be causing aberrant cell death of desirable cells, such as neurons, and one would seek to inhibit the activation of these pathways by turning this signaling down. As such, it would be desired to reduce the amount of misprocessed RNA by procuring compounds that can either separate mis-splicing from activation of antiviral pathways or that reduce the accumulation of aberrant RNAs. In such a case, methods to identify useful compounds would not inhibit RNA helicases or splicing regulators that would cause accumulation of aberrant RNAs.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the systems and methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Cellular and Computational Platform for Screening for Selective RNA Metabolism Modulators

In particular embodiments of systems and methods of the disclosure, prior to subjecting one or more candidate inhibitors to one or more RNA helicases and/or one or more splicing regulators, the function of one or more RNA helicases and/or one or more splicing regulators may be characterized. Given that one or more RNA helicases and/or one or more splicing regulators may be essential for a cell, and therefore a knock-out of these RNA helicases and/or splicing regulators would be cytotoxic, in specific embodiments the function of the RNA helicases and/or splicing regulators are characterized in a manner other than methods like CRISPR. In specific embodiments, loss-of-function of a particular RNA helicase is characterized to assess acute changes using dosage and temporal control of the helicase. In certain cases, one or more desired RNA helicases are subject to biochemical processes to reduce the levels of the helicase protein(s) in a cell, and in a specific case the helicase protein(s) are subject to degradation by enzymatic processes, such as ubiquitination. In one specific example, the RNA helicase(s) are characterized using targeted, CRBN-mediated degradation approaches in which heterobifunctional small molecules that induce dimerization of FK506 binding protein 12 (FKBP12)^{F36V} fusion proteins and the E3 ubiquitin ligase (CRBN) complex, leading to CRBN-mediated degradation of the RNA helicase (see Nabet et al., 2018).

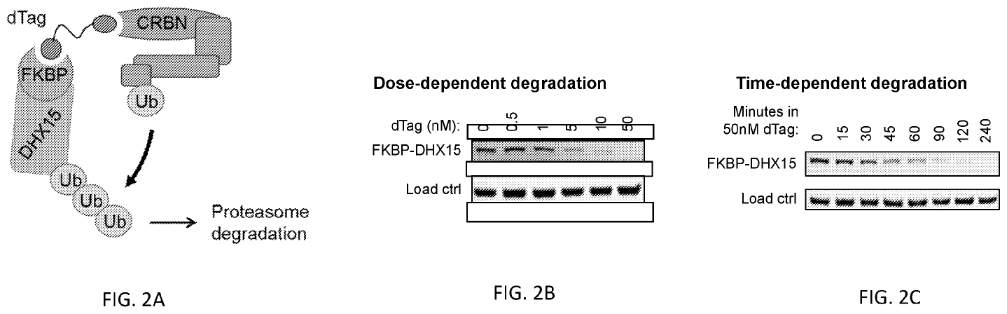

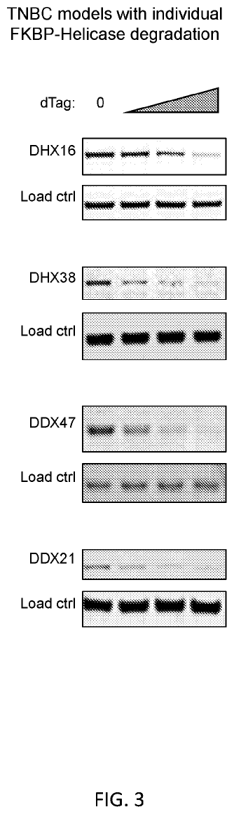

FIG. 2A provides one example of a degradation assay that may be employed to reduce levels of RNA helicases as part of their functional characterization. An RNA helicase of interest (here, DHX15) is fused with FKBP12^{F36V}. Upon treatment with a heterobifunctional small molecule (dTag), the CRBN E3 ubiquitin ligase is recruited to the aforementioned fusion protein leading to its ubiquitination. The ubiquitinated fusion protein is then targeted for degradation by the proteasome. FIG. 2B provides a western blot analysis demonstrating dose-dependent degradation of a fusion of FKBP12^{F36V} and DHX15, per the assay, whereas FIG. 2C is a time-dependent degradation representation for the same fusion. FIG. 3 demonstrates a broad application of the same degradation assay across a variety of RNA helicases in triple negative breast cancer (TNBC) cell lines. The degradation was applied to the examples of DHX16, DHX38, DDX47, and DDX21 RNA helicases. Such a degradation assay is applicable to any RNA helicase, including those specifically listed herein.

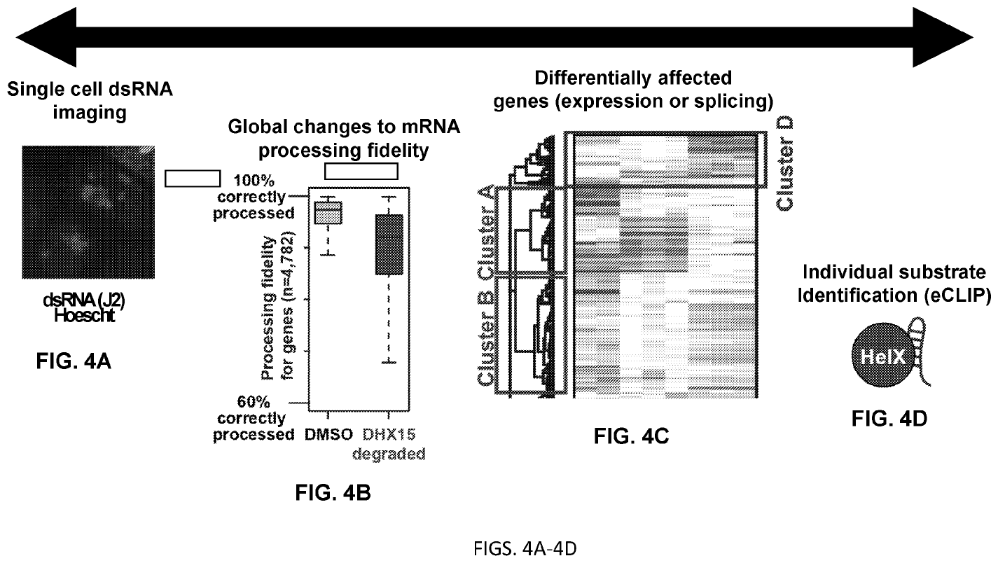

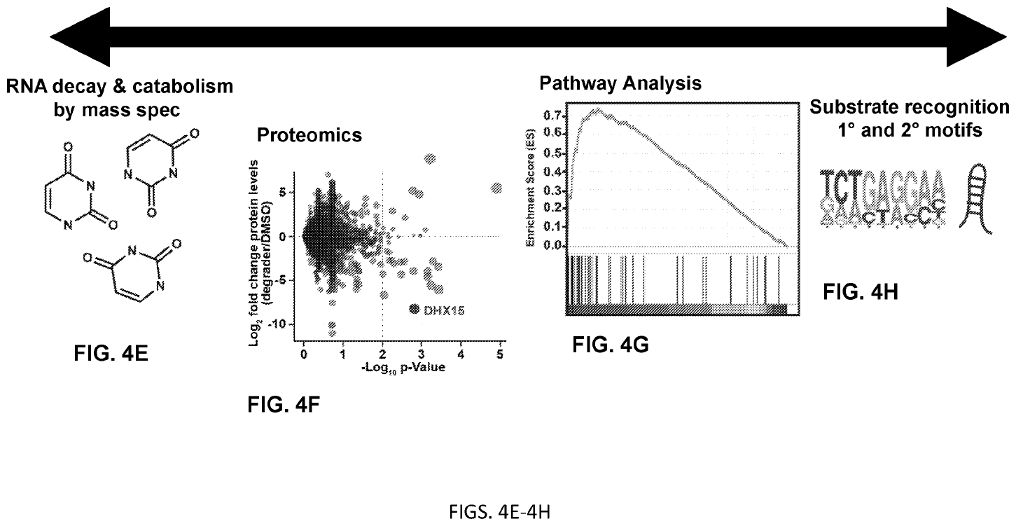

The functional characterization of particular RNA helicases upon their degradation may occur by any suitable methods(s). Some of the assays that measure the outcome of degradation of particular RNA helicases may provide information with respect to global proteomic or RNA changes, whereas others of the assays may provide information more related to event-specific RNA changes. Embodiments of the disclosure include integrated assays for multi-scale RNA phenotyping and target mechanism of action (MOA). As examples only, FIGS. 4A-4H illustrate a variety of assays for characterizing RNA phenotype following degradation of one or more specific RNA helicases to establish its effect in a cell. The assays cover the spectrum of impacting global RNA changes in a cell to impacting event-specific RNA changes at a more local level. Examples of assays include the following: single cell dsRNA imaging (FIG. 4A); global changes to mRNA processing fidelity (FIG. 4B); differentially affected genes (expression or splicing) (FIG. 4C); individual substrate identification (eCLIP) (FIG. 4D); RNA decay and catabolism by mass spectrometry (FIG. 4E), proteomics (FIG. 4F), pathway analysis (FIG. 4G), and substrate recognition for primary and secondary motifs (FIG. 4H).

Single cell dsRNA imaging as demonstrated in FIG. 4A may be utilized to measure for changes upon degradation of the RNA helicase with respect to accumulation of double stranded RNAs not normally present in cells. In certain cases, such imaging may utilize the methods of Bowling et al. (2021), in which immunofluorescence with an antibody that recognizes 40 base pair long dsRNA is performed. In cells in which a particular RNA helicase is degraded, one can compare dsRNA imaging to control cells lacking such degradation.

FIG. 4B shows an example for DHX15 degradation to ascertain global changes to mRNA processing fidelity. The processing fidelity of thousands of mRNAs is measured in cells having the degradation vs. cells that do not have the degradation. The vast number of genes being measured provides a fingerprint for global changes rather than a localized fingerprint of only a few genes. One can also utilize methods of measuring RNA decay and catabolism products using mass spectrometry to provide large-scale information of the impact of RNA helicase degradation on these processes (FIG. 4E). One can also utilize proteomics methods to identify changes to the proteome associated with RNA helicase loss of function and/or degradation. FIG. 4F shows an example of proteomics analysis from cells in which DHX15 was degraded.

In some embodiments following RNA helicase degradation, differentially affected genes with respect to expression or splicing are measured in cells, allowing a fingerprint of a number of genes (FIG. 4C). In such an assay, there is analysis from RNAseq data seeking genes whose expression levels and/or splicing status change upon helicase degradation. Although in at least some cases the expression of the majority of, virtually all, or all genes are examined in the assay, not all are differentially changed, and this produces a signature of changes associated with helicase loss of function/degradation.

With respect to measuring more localized RNA changes, one can employ individual substrate identification (eCLIP) (FIG. 4D). In an eCLIP assay, a protein of interest is crosslinked to its RNA substrates, and the protein is immunoprecipitated with the associated RNA being isolated and sequenced. The resultant data provides information regarding RNAs for which the helicase can bind. In other cases, one may utilize pathway analysis (FIG. 4G) and/or substrate recognition or primary and secondary motifs (FIG. 4H). Such assays give information about specific sequence(s) of RNA for interaction with RNA binding proteins, including helicases, and/or give information about specific conformation/structure for interaction of RNA with RNA binding proteins, including helicases. Thus, the RNAseq and eCLIP data in combination allows identification of motifs required for binding by a helicase of interest.

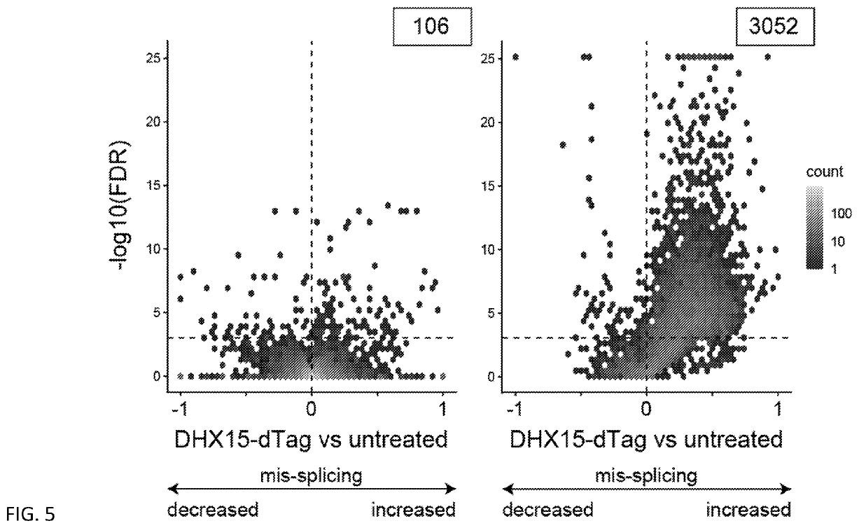

Particular embodiments of the disclosure provide computational approaches for identifying RNA helicase fingerprints and PD biomarker discovery. In certain embodiments, the sensitivity of the methods is encompassed in algorithms for detection of RNA mis-splicing. In at least some cases, the methods employ directed acyclic graph theory for analysis of RNA splicing. The employed methods allow for 30-100-fold better sensitivity in detecting RNA mis-splicing compared to known methods in the art. In FIG. 5, for example, known methods in the art are shown on the left, and the computational methods of the present disclosure are shown on the right. Each plot is a comparison of the change in RNA processing fidelity in DHX15-degraded cells compared to DHX15 wildtype cells such that the comparisons for the method in the art and the method of the present disclosure are of cells having the same conditions. The computational methods of the present disclosure were able to identify 3052 mis-spliced transcripts (in this example, this refers to 3052 transcripts each having at least one misspliced intron), compared to methods in the art that could identify only 106 mis-spliced transcripts.

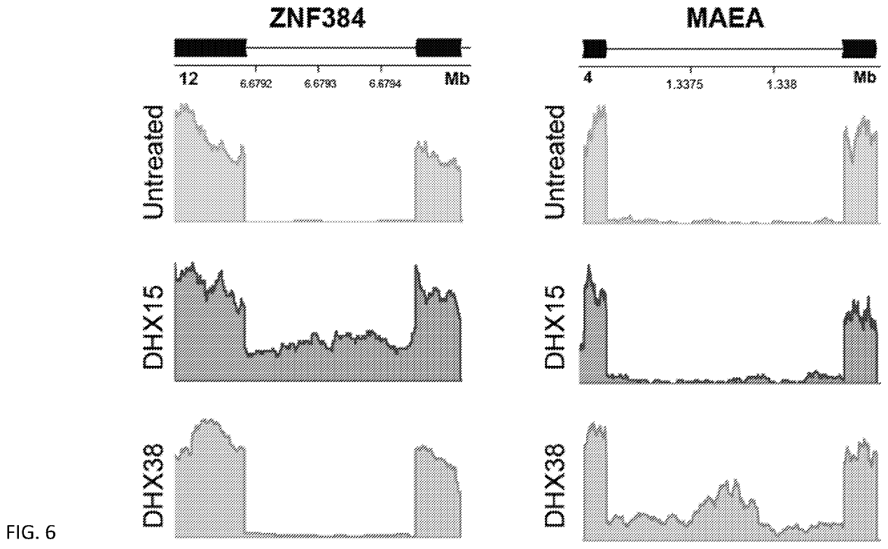

FIG. 6 provides an example of selectivity identified by the computational embodiments toward the identification of fingerprints of selective inhibition of RNA helicases. FIG. 6 demonstrates selective mis-splicing of two representative genes (ZNF384 and MAEA) upon inhibition of two exemplary RNA helicases. The gene map illustrates one intron in the respective genes, and the representations below demonstrate the quality of RNA splicing upon degradation of DHX15 vs. degradation of DHX38. With degradation of DHX15, splicing of ZNF384 is impacted but not splicing of MAEA. With degradation of DHX38, splicing of MAEA is impacted but not splicing of ZNF384. Such differential effect on helicases may be extrapolated to many genes and for many RNA helicases, including those described herein.

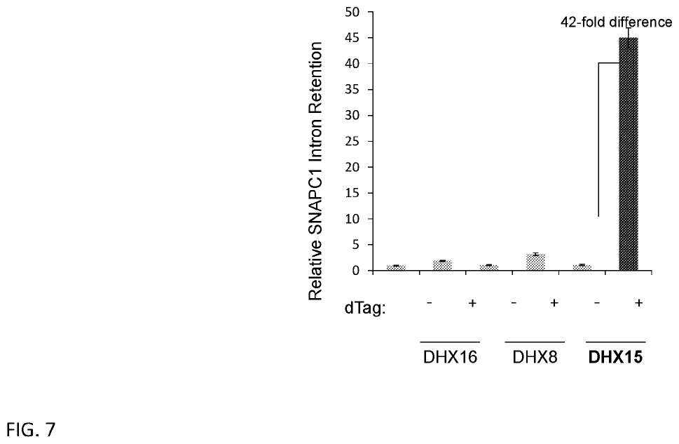

In some embodiments, computational approaches identify biomarkers, including selective PD markers of particular RNA helicase activity (FIG. 7). Specific aspects of such methods provide for PD assays for individual or combinatorial helicase inhibition. Selective substrates may be used for in vitro assay cascades for RNA binding and unwinding. As such, FIG. 7 demonstrates the relative intron retention for the SNAPC1 gene (as an example) in cell lines in which DHX16, DHX8, or DHX15 have been degraded. For this particular gene, degradation of DHX15 had a deleterious impact on RNA splicing, but not for degradation of DHX16 or DHX8. Such differential effect on helicases may be extrapolated to many genes and for many RNA helicases, including those described herein.

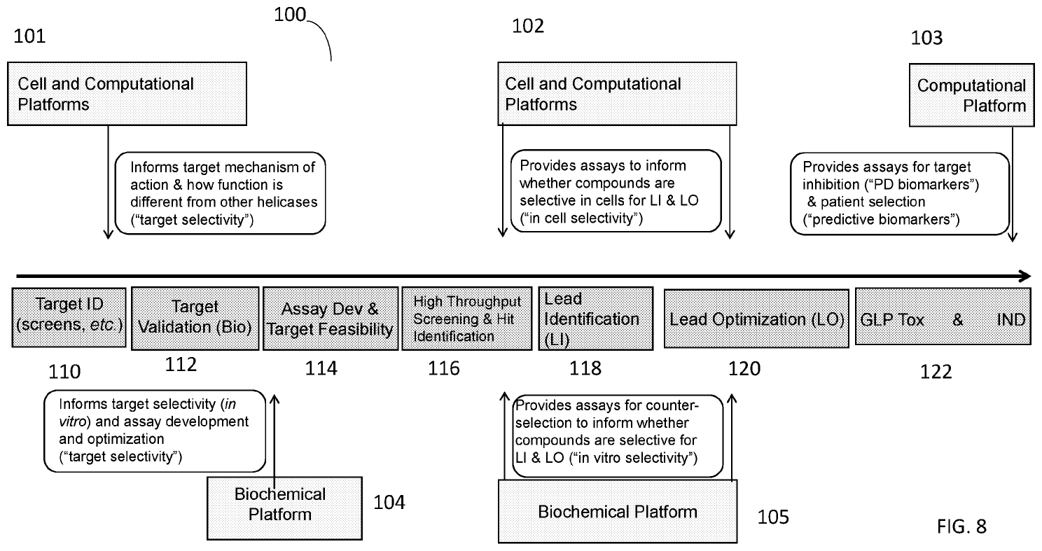

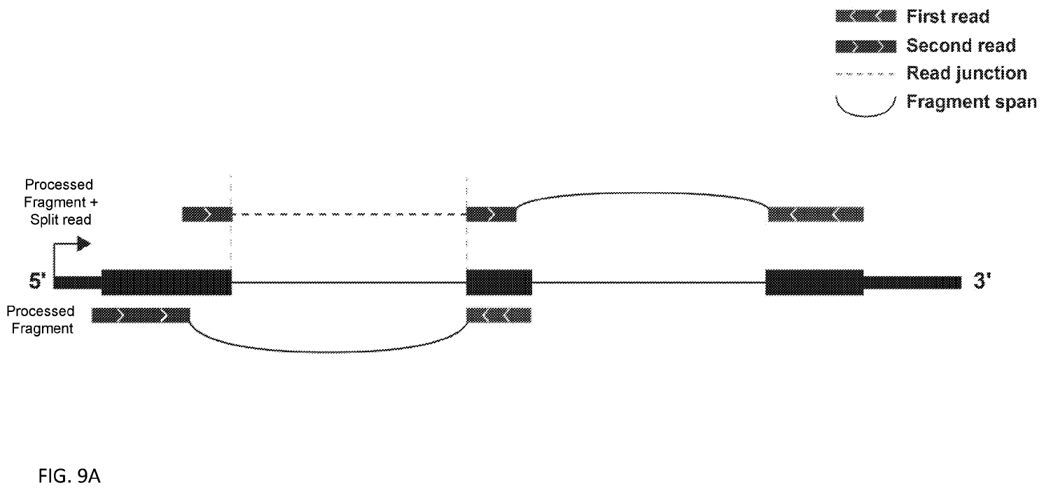

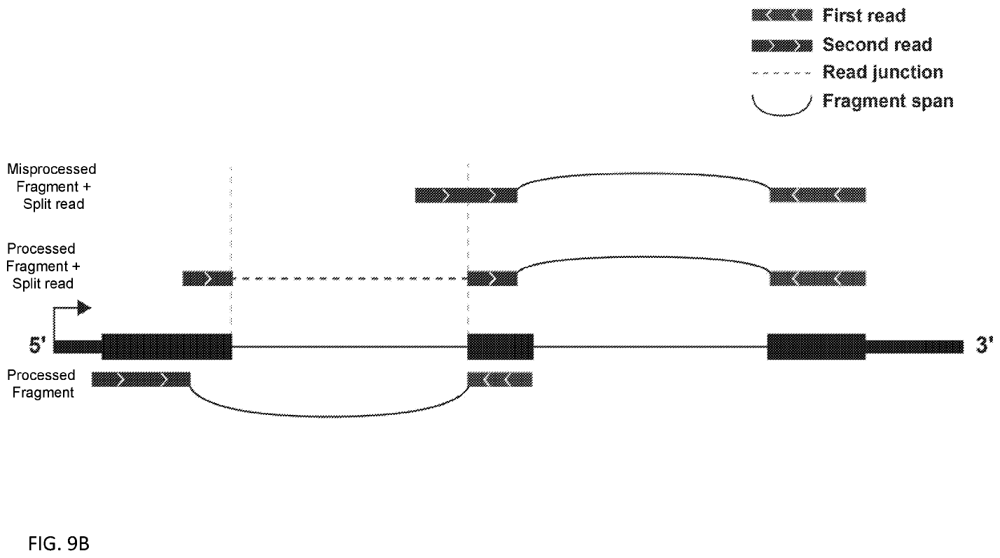

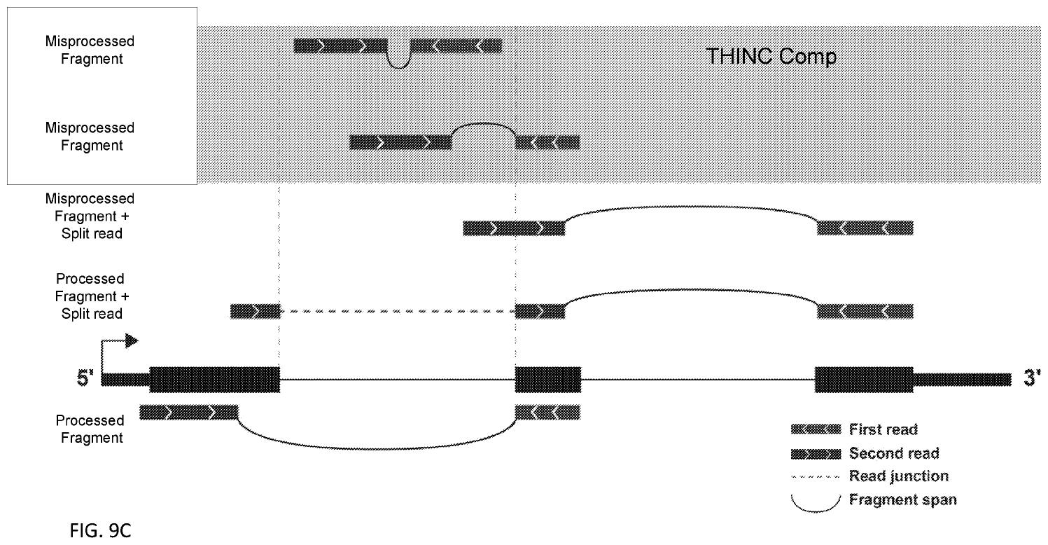

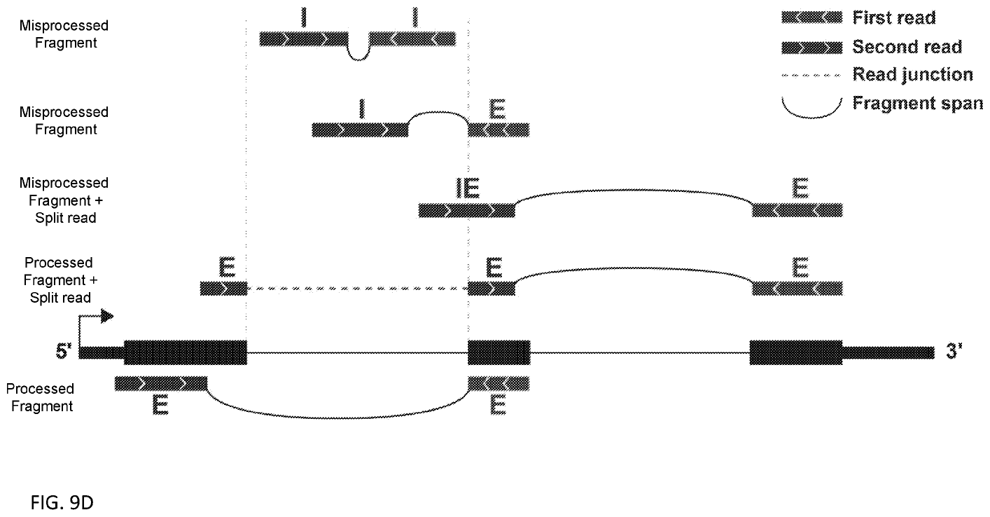

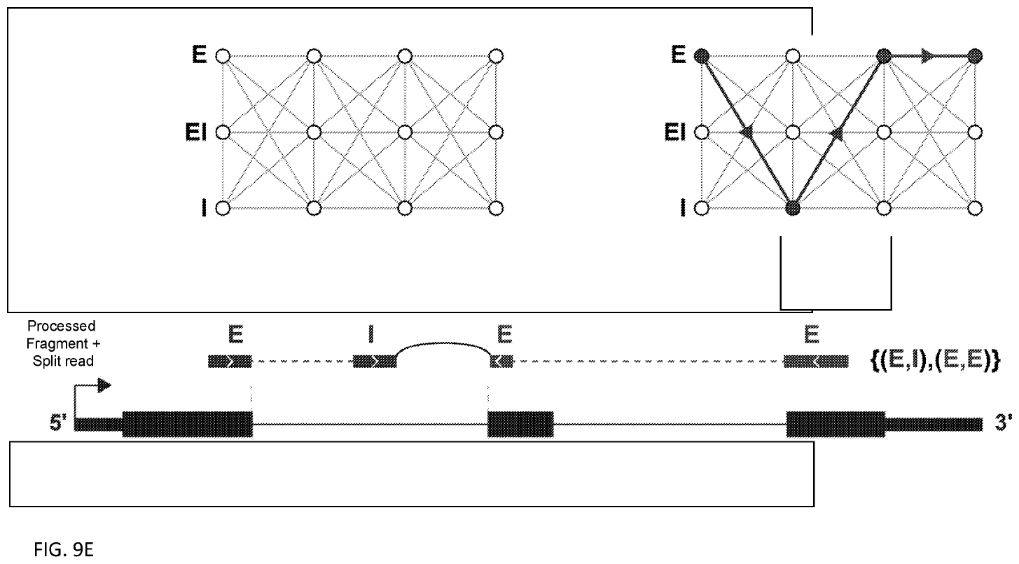

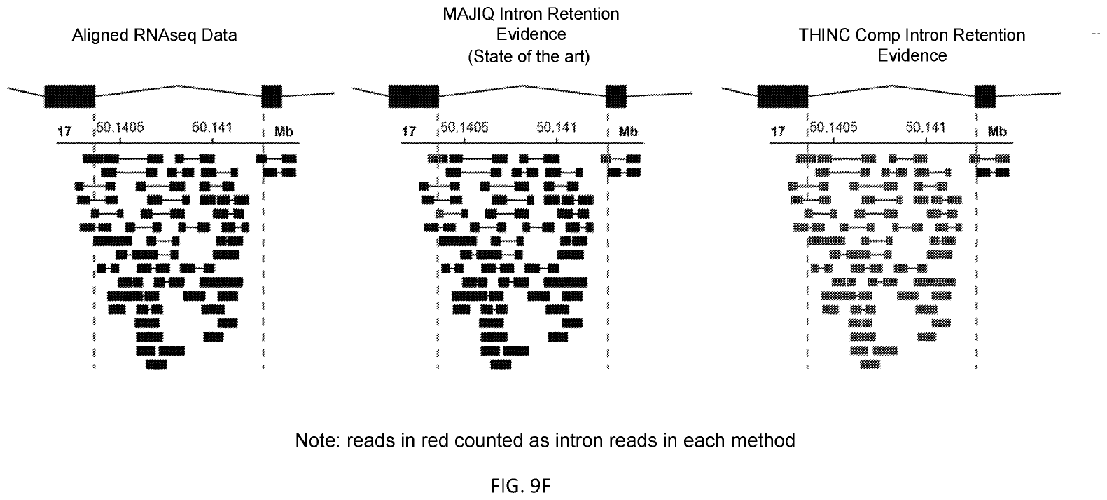

FIG. 8 provides one example of a system 100 for developing or identifying compounds useful for manipulating processes associated with RNA metabolism. In specific cases, the system 100 is utilized for identifying or screening for inhibitors against a class of proteins related to RNA metabolism, such as RNA helicases or splicing regulators, in which the system includes selectivity profiling throughout multiple steps or stages, in at least some cases. Although in specific embodiments the order of steps or actions are successive in nature from left to right of the image, in alternative embodiments the order of steps or actions are modified compared to this order. For example, one step or action may occur before a subsequent step or action as depicted in FIG. 8, but in alternative embodiments an order of succession is reversed. In some cases, one or more steps or actions may occur at substantially the same time. In particular embodiments, FIG. 8 illustrates a system 100 for identifying inhibitors of RNA helicases and/or splicing regulators in which the respective RNA helicases and/or splicing regulators are selectively profiled so that they target one or more respective particular helicases and/or splicing regulators but also that they do not target other proteins, including other respective helicases and/or splicing regulators. Such an aspect of the system may be utilized because inhibition of the excluded helicases and/or splicing regulators would be toxic to a cell or tissue or organ or individual. In particular embodiments, the system of the present disclosure is an in-cell or in vivo system.

In a specific example of an embodiment of the system 100 of FIG. 8, the initial one or two steps or actions incorporate cellular and/or computational platforms 101 that inform for a particular one or more helicases a target mechanism of action and/or how the one or more particular helicases function differently from others, and this may be considered target selectivity. Such initial one or two steps or actions may include target identification 110, such as by cellular screens, and so forth. The target identification 110 may identify one target, or a plurality of related targets, for which inhibition may be desirable, such as may be useful for a clinical application. The screens may assay for one or more structural characteristics and/or one or more functional characteristics of one or a plurality of potential target proteins, but in specific embodiments the assay provides information at least on a target mechanism of action. In particular embodiments, target identification 110 comprises disruption of one or more RNA helicases and/or one or more splicing regulators individually or as a pool and analysis of the pertinent normal or cancer cell phenotype(s) which is in some instances growth. In some cases, disruption of one or more RNA helicases and/or one or more splicing regulators has no substantive impact on growth, and this may or may not be informative. In other cases, disruption of one or more RNA helicases and/or one or more splicing regulators enhances or impairs growth. The pertinent phenotype may be measured by any suitable methods, including cell counts, for example. Following target identification 110, there may be a target validation step 112 in which the functional and/or structural characteristics of the identified targets in target identification step 110 are validated through biological means. In some cases, biological validation of a particular target or targets includes target perturbation followed by cell growth, cell death, cell signaling and/or RNA-based and protein-based measurements to discern target mechanism of action in the appropriate cellular context in vitro or in vivo. These studies provide further evidence that target inhibition may be desirable in a particular clinical context and provide information that may be utilized as pharmacodynamic markers of target inhibition.

Following one to two steps or actions utilizing cellular and computational platforms, the next one or more steps may implement biochemical platform 104 in which information about target selectivity in vitro is obtained and target selectivity may be maintained for assay development and optimization or feasibility step 114 for one or a plurality of target proteins, including RNA helicases and/or splicing regulators. Thus, in this stage as well, target selectivity is performed, in certain embodiments. As a result of the assay development and optimization of feasibility step 116, there may be high throughput screening (HTS) for a plurality of test candidates that inhibit one or more desired proteins. The test candidates that show favorable characteristics in the HTS may be identified. These characteristics include tractable chemistry, inhibition of activity when treated with 10 uM, and no discernable chemical liabilities.

The test candidate inhibitors that are the outcome of steps 110, 112, 114, and 116 may be considered lead compound(s) and may be subject to additional cell and computational platforms 102 and biochemical platform 105 for enhanced characterization. The cell and computational platforms 102 provide assays to obtain information whether compounds are selective in cells. The biochemical platform 105, which may occur at substantially the same time as cell and computational platforms 102, may provide in vitro assays for counter-selection to provide information whether the compounds are selective or maintain selectivity. Following this, the lead compound(s) may be subject to computational platform 103 in which assays are utilized for target inhibition to identify PD biomarkers and/or to identify predictive biomarkers for selection of recipient individuals for which the lead compound(s) would be therapeutically effective.

### REFERENCES

All publications cited herein are hereby incorporated by reference in their entirety herein.
Bowling, Elizabeth A., et al. Spliceosome-targeted therapies trigger an antiviral immune response in triple-negative breast cancer. Cell volume 184, pages 1-20 (2021).
Nabet, Behnam, et al. The dTAG system for immediate and target-specific protein degradation. Nature Chemical Biology volume 14, pages 431-441 (2018).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of screening for a selective inhibitor of one or more RNA helicases or one or more splicing regulators, comprising:
providing an in-cell or in vivo assay for disruption of one or more RNA helicases or one or more splicing regulators;
measuring one or more RNA metabolism parameters and/or proteomics in the cell following said disruption;
subjecting one or more candidate inhibitors to the in-cell or in vivo assay for each RNA helicase in a plurality of helicases for identification of candidate inhibitors that modify the one or more RNA metabolism parameters and/or proteomics for a first subset of RNA helicases and/or splicing regulators in the plurality but that do not modify the one or more RNA metabolism parameters and/or splicing regulators for a second subset of RNA helicases in the plurality.

2. The method of claim 1, wherein the disruption is at the gene level or at the mRNA or protein level.

3. The method of claim 2, wherein the disruption is at the protein level.

4. The method of claim 3, wherein the disruption is carried out by enzymatic degradation, small molecule inhibition of enzymatic activity, allosteric small molecule inhibitor, or small molecule degrader.

5. The method of claim 4, wherein the enzymatic degradation is degradation by the proteasome.

6. The method of claim 2, wherein the disruption is at the gene level or at the mRNA level.

7. The method of claim 6, wherein the disruption is carried out by RNAi, siRNA, shRNA, ribozymes, CRISPRCas9, homologous recombination, site-specific nucleases, zinc fingers, or TALENs.

8. The method of any one of the preceding claims, wherein the cells of the in-cell assay are normal.

9. The method of any one of claims 1-8, wherein the cells are not cancerous.

10. The method of any one of claims 1-8, wherein the cells of the in-cell assay are cancerous.

11. The method of claim 10, wherein the cancerous cells are breast cancer, non-small cell lung cancer, small cell lung cancer, colorectal cancer, subtypes of leukemia, subtypes of lymphoma, ovarian cancer, esophageal cancer, hepatic cell carcinoma, head and neck cancer, gastric cancer, pancreatic cancer, or bladder cancer.

12. The method of any one of the preceding claims, wherein the one or more RNA metabolism parameters is RNA splicing, RNA decay, RNA catabolism, RNA export, transcription, translation, rRNA biogenesis, RNA modification, or a combination thereof.

13. The method of any one of the preceding claims, wherein the measuring step comprises measuring double stranded RNA, mRNA processing, differential expression, differential splicing, global RNA processing fidelity, RNA catabolites, protein levels, RNA substrates, RNA binding motifs, or a combination thereof.

14. The method of any one of the preceding claims, wherein the measuring step comprises measuring intron splicing in one or more genes upon degradation of one or more RNA helicases.

15. The method of any one of the preceding claims, wherein the measuring step comprises measuring intron splicing in multiple genes upon degradation of multiple RNA helicases.

16. The method of claim 15, wherein information from measuring intron splicing in multiple genes upon degradation of multiple RNA helicases results in said identification of inhibitors that modify RNA splicing for a first subset of RNA helicases in the plurality but that do not modify RNA splicing for a second subset of RNA helicases in the plurality.

17. The method of claim 16, further comprising the step of formulating said inhibitors in a pharmaceutically acceptable carrier.

18. The method of claim 1, wherein the subjecting step is further defined as:
subjecting one or more candidate inhibitors to the in-cell assay for each RNA helicase in the plurality to identify candidate inhibitors that modify the one or more parameters of RNA metabolism or proteomics for a first subset of RNA helicases in the plurality, followed by identifying the absence of modifying the one or more parameters of RNA metabolism or proteomics for the second subset of RNA helicases in the plurality.

19. The method of claim 1, wherein the subjecting step is further defined as:
subjecting one or more candidate inhibitors to the in-cell assay for each RNA helicase in the plurality to identify candidate inhibitors that do not modify the one or more parameters of RNA metabolism or proteomics for the second subset of RNA helicases in the plurality, followed by identifying the presence of modifying for the one or more parameters of RNA metabolism or proteomics for the first subset of RNA helicases in the plurality.

20. The method of claim 1, wherein the subjecting step is further defined as:
subjecting one or more candidate inhibitors to the in-cell assay for each RNA helicase in the plurality to identify candidate inhibitors that modify the one or more parameters of RNA metabolism or proteomics for a first subset of RNA helicases in the plurality at substantially the same time as identifying the absence of modifying the one or more parameters of RNA metabolism or proteomics for the second subset of RNA helicases in the plurality.

21. The method of any one of the preceding claims, wherein the candidate inhibitors are small molecules, proteins, peptides, nucleic acid, carbohydrate, or a combination thereof.

22. The method of claim 21, wherein the candidate inhibitors are small molecules.

23. The method of any one of the preceding claims, wherein the subjecting step comprises high throughput screening.

24. The method of any one of the preceding claims, wherein the RNA helicases in the first subset of RNA helicases are from the same sub-family of RNA helicases.

25. The method of any one of the preceding claims, wherein the RNA helicases in the first subset of RNA helicases share the same function in RNA metabolism.

26. The method of claim 25, wherein the function is RNA splicing.

27. The method of any one of the preceding claims, wherein the first subset of RNA helicases comprises one or more RNA helicases.

28. The method of any one of the preceding claims, wherein the second subset of RNA helicases comprises one or more RNA helicases.

29. The method of claim 26, wherein the RNA splicing by the RNA helicase(s) in the first subset of RNA helicases is aberrant in cancer cells.

30. The method of any one of the preceding claims, wherein the plurality of RNA helicases comprises two or more of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

31. The method of claim 30, wherein the plurality of RNA helicases comprises the majority of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

32. The method of claim 30, wherein the plurality of RNA helicases comprises all of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

33. The method of any one of the preceding claims, wherein the plurality of RNA helicases comprises DHX15.

34. The method of any one of the preceding claims, wherein the first subset of RNA helicases comprises DHX15.

35. The method of any one of the preceding claims, wherein identification of candidate inhibitors that modify the one or more RNA metabolism parameters for a first subset of RNA helicases in the plurality but that do not modify the one or more RNA metabolism parameters for a second subset of RNA helicases in the plurality is performed by a computer.

36. The method of any one of the preceding claims, wherein identification of candidate inhibitors that modify the one or more RNA metabolism parameters for a first subset of RNA helicases in the plurality but that do not modify the one or more RNA metabolism parameters for a second subset of RNA helicases in the plurality utilizes an algorithm.

37. A method of predicting toxicity of a compound for an individual, comprising the steps of:
providing the compound to a non-diseased cell and identifying a first RNA signature for the cell; and
comparing the first RNA signature to a second RNA signature associated with disruption of an RNA helicase or splicing regulator in a cell, wherein said disruption is toxic to a cell or organism,
wherein when the first RNA signature and the second RNA signature are the same or are substantially the same, the compound is predicted to be toxic for the individual, and wherein when the first RNA signature and the second RNA signature are not the same or not substantially the same, the compound is predicted not to be toxic for the individual.

38. The method of claim 37, further defined as:
comparing the first RNA signature to a plurality of other RNA signatures each respectively associated with disruption of an RNA helicase or a splicing regulator, wherein said disruption of a subset of the plurality of other RNA helicases and/or splicing regulators is toxic to a cell or organism; and
wherein when the first RNA signature and the RNA signatures of the subset are the same or are substantially the same, the compound is toxic for the individual, and wherein when the first RNA signature and the RNA signatures of the subset are not the same or not substantially the same, the compound is not toxic for the individual.

39. The method of claim 38, wherein the comparing step is performed by a computer.

40. The method of claim 38 or 39, wherein the comparing step is performed with an algorithm.

41. The method of any one of claims 37-40, wherein the RNA signature from a disruption of an RNA helicase or splicing regulator comprises misprocessed RNA.

42. The method of any one of claims 38-41, further comprising the step of producing the disruption of the RNA helicase or a splicing regulator.

43. The method of claim 42, wherein the disruption is at the gene level or at the mRNA or protein level.

44. The method of claim 43, wherein the disruption is at the protein level.

45. The method of claim 44, wherein the disruption is carried out by enzymatic degradation, small molecule inhibition of enzymatic activity, allosteric small molecule inhibitor, or small molecule degrader.

46. The method of claim 45, wherein the enzymatic degradation is degradation by the proteasome.

47. The method of claim 43, wherein the disruption is at the gene level or at the mRNA level.

48. The method of claim 47, wherein the disruption is carried out by RNAi, siRNA, shRNA, ribozymes, CRISPRCas9, homologous recombination, site-specific nucleases, zinc fingers, or TALENs.

49. The method of any one of claims 37-48, wherein the non-diseased cell is a non-cancerous cell of the same type with respect to cancer cells of interest, is a normal peripheral blood mononuclear cell, or is a lymphocyte.

50. A method of screening for an activator of antiviral immune signaling and/or antitumor immune signaling, comprising the steps of:
subjecting cancer cells or normal cells separately to a plurality of candidate activators of antiviral immune signaling and/or antitumor immune signaling;
measuring which candidate activators produce an RNA signature comprising accumulation of misprocessed RNAs in the cells; and
comparing output from the measuring step to RNA signatures produced from one or a plurality of disrupted RNA helicases and/or splicing regulators in normal cells,
wherein when a candidate activator produces an RNA signature comprising accumulation of misprocessed RNAs that is the same as, or substantially the same as, an RNA signature from a disrupted RNA helicase and/or splicing regulator that produces misprocessed RNA that activates antiviral immune signaling and/or antitumor immune signaling, then the candidate activator is an activator of antiviral immune signaling and/or antitumor immune signaling.

51. The method of claim 50, wherein the subjecting step comprises subjecting the plurality to a variety of types of cancer cells and comparing the output between two or more cancer types to RNA signatures produced from the plurality of disrupted RNA helicases and/or splicing regulators.

52. The method of claim 50, wherein the activator is not known to be an inhibitor of an RNA helicase or a splicing regulator or is not an inhibitor of an RNA helicase or a splicing regulator.

53. The method of any one of claims 50-52, wherein the comparing step is performed by a computer.

54. The method of any one of claims 50-53, wherein the comparing step is performed with an algorithm.

55. The method of any one of claims 50-54, wherein the activator is provided in a therapeutically effective amount to an individual in need thereof.

56. The method of claim 55, wherein the individual has cancer or an infectious disease.

57. The method of any one of claims 50-56, further comprising the step of producing the disruption of the disrupted RNA helicases and/or splicing regulators.

58. The method of claim 57, wherein the disruption is at the gene level or at the mRNA or protein level.

59. The method of claim 58, wherein the disruption is at the protein level.

60. The method of claim 59, wherein the disruption is carried out by enzymatic degradation, small molecule inhibition of enzymatic activity, allosteric small molecule inhibitor, or small molecule degrader.

61. The method of claim 60, wherein the enzymatic degradation is degradation by the proteasome.

62. The method of claim 58, wherein the disruption is at the gene level or at the mRNA level.

63. The method of claim 62, wherein the disruption is carried out by RNAi, siRNA, shRNA, ribozymes, CRISPRCas9, homologous recombination, site-specific nucleases, zinc fingers, or TALENs.

64. A method of identifying inhibitors of antiviral immune signaling and/or antitumor immune signaling, comprising the steps of:
subjecting candidate inhibitors to cells and measuring from the cells respective RNA signatures for misprocessed RNA; and
comparing the RNA signatures from the cells in the subjecting step to one or more RNA signatures from cells each comprising disruption of an RNA helicase or a splicing regulator,
wherein when an RNA signature produced in the cells upon subjecting them to a candidate inhibitor comprises a reduced amount of misprocessed RNA and/or comprises a reduced amount of particular misprocessed RNA molecules when compared to the RNA signatures from cells each comprising disruption of an RNA helicase or a splicing regulator, the candidate inhibitor is an inhibitor of antiviral immune signaling and/or antitumor immune signaling.

65. The method of claim 64, wherein the comparing step is performed by a computer.

66. The method of 64 or 66, wherein the comparing step is performed with an algorithm.

67. The method of any one of claims 64-67, wherein the activator is provided in a therapeutically effective amount to an individual in need thereof.

68. The method of claim 68, wherein the individual has cancer or an infectious disease.

69. The method of any one of claims 64-68, further comprising the step of producing the disruption of the RNA helicase or splicing regulator.

70. The method of claim 69, wherein the disruption is at the gene level or at the mRNA or protein level.

71. The method of claim 70, wherein the disruption is at the protein level.

72. The method of claim 71, wherein the disruption is carried out by enzymatic degradation, small molecule inhibition of enzymatic activity, allosteric small molecule inhibitor, or small molecule degrader.

73. The method of claim 72, wherein the enzymatic degradation is degradation by the proteasome.

74. The method of claim 70, wherein the disruption is at the gene level or at the mRNA level.

75. The method of claim 74, wherein the disruption is carried out by RNAi, siRNA, shRNA, ribozymes, CRISPRCas9, homologous recombination, site-specific nucleases, zinc fingers, or TALENs.

76. A method of screening for an inhibitor of antiviral immune signaling and/or antitumor immune signaling, comprising the steps of:
subjecting cancer cells or normal cells separately to a plurality of candidate inhibitors of antiviral immune signaling and/or antitumor immune signaling;
measuring which candidate inhibitors produce an RNA signature comprising accumulation of misprocessed RNAs in the cells; and
comparing output from the measuring step to RNA signatures produced from one or a plurality of disrupted RNA helicases and/or splicing regulators in normal cells,
wherein when a candidate inhibitor produces an RNA signature comprising accumulation of misprocessed RNAs that is the same as, or substantially the same as, an RNA signature from a disrupted RNA helicase and/or splicing regulator that produces misprocessed RNA that activates antiviral immune signaling and/or antitumor immune signaling, then the candidate inhibitor is an inhibitor of antiviral immune signaling and/or antitumor immune signaling.

77. The method of claim 76, wherein the comparing step is performed by a computer.

78. The method of claim 76 or 77, wherein the comparing step is performed with an algorithm.

79. The method of any one of claims 76-78, wherein the inhibitor is provided in a therapeutically effective amount to an individual in need thereof.

80. The method of claim 79, wherein the individual has neurodegeneration or an autoimmune disease.

81. The method of any one of claims 78-80, further comprising the step of producing the disruption of the RNA helicases and/or splicing regulators.

82. The method of claim 81, wherein the disruption is at the gene level or at the mRNA or protein level.

83. The method of claim 82, wherein the disruption is at the protein level.

84. The method of claim 83, wherein the disruption is carried out by enzymatic degradation, small molecule inhibition of enzymatic activity, allosteric small molecule inhibitor, or small molecule degrader.

85. The method of claim 84, wherein the enzymatic degradation is degradation by the proteasome.

86. The method of claim 82, wherein the disruption is at the gene level or at the mRNA level.

87. The method of claim 86, wherein the disruption is carried out by RNAi, siRNA, shRNA, ribozymes, CRISPRCas9, homologous recombination, site-specific nucleases, zinc fingers, or TALENs.

88. A method of treating a subject having a medical condition associated with defective RNA metabolism comprising administering to the subject a pharmaceutical composition comprising a therapeutically effective amount of a selective inhibitor of one or more RNA helicases,
wherein the inhibitor is identified by an in vivo screening method comprising the steps of:
providing an in-cell or in vivo assay for disruption of one or more RNA helicases or one or more splicing regulators;
measuring one or more RNA metabolism parameters including RNA transcripts and/or proteomics in the cell following said disruption; and
subjecting one or more test candidates to the in-cell or in vivo assay for each RNA helicase in a plurality of helicases for identification of test candidates that modify the one or more RNA metabolism parameters and/or proteomics for a first subset of RNA helicases and/or splicing regulators in the plurality but that do not modify the one or more RNA metabolism parameters and/or splicing regulators for a second subset of RNA helicases in the plurality.

89. The method of claim 88, wherein the defective RNA metabolism is defective synthesis, folding/unfolding, modification, processing, stabilization, or degradation of RNA.

90. The method of claim 88 or 89, wherein the defective RNA metabolism is accumulation of misprocessed RNA.

91. The method of any one of claims 88-90, wherein the defective RNA metabolism is defective RNA splicing.

92. The method of claim 88, wherein the disruption is at the gene level or at the mRNA or protein level.

93. The method of claim 92, wherein the disruption is at the protein level.

94. The method of claim 93, wherein the disruption is carried out by enzymatic degradation, small molecule inhibition of enzymatic activity, allosteric small molecule inhibitor, or small molecule degrader.

95. The method of claim 94, wherein the enzymatic degradation is degradation by the proteasome or phagosome.

96. The method of claim 92, wherein the disruption is at the gene level or at the mRNA level.

97. The method of claim 96, wherein the disruption is carried out by RNAi, siRNA, shRNA, ribozymes, CRISPRCas9, homologous recombination, site-specific nucleases, zinc fingers, or TALENs.

98. The method of any one of claims 88-97, wherein the cells of the in-cell assay are normal.

99. The method of any one of claims 88-98, wherein the cells are not cancerous.

100. The method of any one of claims 88-99, wherein the cells of the in-cell assay are cancerous.

101. The method of claim 100, wherein the cancerous cells are breast cancer, non-small cell lung cancer, small cell lung cancer, colorectal cancer, all subtypes of leukemia, all subtypes of lymphoma, ovarian cancer, esophageal cancer, hepatic cell carcinoma, head and neck cancer, gastric cancer, pancreatic cancer, or bladder cancer.

102. The method of any one of claims 88-101, wherein the one or more RNA metabolism parameters is RNA splicing, RNA decay, RNA catabolism, RNA export, transcription, translation, rRNA biogenesis, RNA modification, or a combination thereof.

103. The method of any one of claims 88-102, wherein the measuring step comprises measuring double stranded RNA, mRNA processing, differential expression, differential splicing, global RNA processing fidelity, RNA catabolites, protein levels, RNA substrates, RNA binding motifs, or a combination thereof.

104. The method of any one of claims 88-103, wherein the measuring step comprises measuring intron splicing in one or more genes upon degradation of one or more RNA helicases.

105. The method of any one of claims 88-104, wherein the measuring step comprises measuring intron splicing in multiple genes upon degradation of multiple RNA helicases.

106. The method of claim 105, wherein information from measuring intron splicing in multiple genes upon degradation of multiple RNA helicases results in said identification of inhibitors that modify RNA splicing for a first subset of RNA helicases in the plurality but that do not modify RNA splicing for a second subset of RNA helicases in the plurality.

107. The method of claim 106, further comprising the step of formulating said inhibitors in a pharmaceutically acceptable carrier.

108. The method of claim 88, wherein the subjecting step is further defined as:
subjecting one or more test candidates to the in-cell assay for each RNA helicase in the plurality to identify test candidates that modify the one or more parameters of RNA metabolism or proteomics for a first subset of RNA helicases in the plurality, followed by identifying the absence of modifying the one or more parameters of RNA metabolism or proteomics for the second subset of RNA helicases in the plurality.

109. The method of claim 88, wherein the subjecting step is further defined as:
subjecting one or more test candidates to the in-cell assay for each RNA helicase in the plurality to identify test candidates that do not modify the one or more parameters of RNA metabolism or proteomics for the second subset of RNA helicases in the plurality, followed by identifying the presence of modifying for the one or more parameters of RNA metabolism or proteomics for the first subset of RNA helicases in the plurality.

110. The method of claim 88, wherein the subjecting step is further defined as:
subjecting one or more test candidates to the in-cell assay for each RNA helicase in the plurality to identify test candidates that modify the one or more parameters of RNA metabolism or proteomics for a first subset of RNA helicases in the plurality at substantially the same time as identifying the absence of modifying the one or more parameters of RNA metabolism or proteomics for the second subset of RNA helicases in the plurality.

111. The method of any one of claims 88-110, wherein the test candidates are small molecules, proteins, peptides, nucleic acid, carbohydrate, or a combination thereof.

112. The method of claim 111, wherein the test candidates are small molecules.

113. The method of any one of claims 88-112, wherein the subjecting step comprises high throughput screening.

114. The method of any one of claims 88-113, wherein the RNA helicases in the first subset of RNA helicases are from the same sub-family of RNA helicases.

115. The method of any one of claims 88-114, wherein the RNA helicases in the first subset of RNA helicases share the same function in RNA metabolism.

116. The method of claim 115, wherein the function is RNA splicing.

117. The method of any one of claims 88-116, wherein the first subset of RNA helicases comprises one or more RNA helicases.

118. The method of any one of claims 88-117, wherein the second subset of RNA helicases comprises one or more RNA helicases.

119. The method of claim 115, wherein the RNA splicing by the RNA helicase(s) in the first subset of RNA helicases is aberrant in cancer cells.

120. The method of any one of claims 88-119, wherein the plurality of RNA helicases comprises two or more of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

121. The method of claim 120, wherein the plurality of RNA helicases comprises the majority of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

122. The method of claim 120, wherein the plurality of RNA helicases comprises all of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

123. The method of any one of claims 88-122, wherein the plurality of RNA helicases comprises DHX15.

124. The method of any one of claims 88-123, wherein the first subset of RNA helicases comprises DHX15.

125. The method of any one of claims 88-124, wherein identification of test candidates that modify the one or more RNA metabolism parameters for a first subset of RNA helicases in the plurality but that do not modify the one or more RNA metabolism parameters for a second subset of RNA helicases in the plurality is performed by a computer.

126. The method of any one of claims 88-125, wherein identification of test candidates that modify the one or more RNA metabolism parameters for a first subset of RNA helicases in the plurality but that do not modify the one or more RNA metabolism parameters for a second subset of RNA helicases in the plurality utilizes an algorithm.

127. The method of any one of claims 88-126, wherein the medical condition is cancer.

128. The method of any one of claims 88-127, wherein the cancer is a solid tumor or a hematological tumor.

129. The method of any one of claims 88-128, wherein the inhibitor targets one or more defective RNA helicases that are associated with chemo-refractory malignancies.

130. The method of any one of claims 88-129, wherein the cancer is selected from the group consisting of non-small cell lung cancer adenocarcinoma, ovarian cancer, esophageal cancer, HCC, head and neck cancer, non-small cell lung squamous cancer, breast cancer (including at least triple-negative), gastric cancer, pancreatic cancer, bladder cancer, colon cancer, cecum cancer, stomach cancer, brain cancer, kidney cancer, larynx cancer, sarcoma, lung cancer, melanoma, prostate cancer, tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

131. The method of any one of claims 88-130, wherein the cancer is a histological type comprising neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

132. The method of any one of claims 88-131, wherein the medical condition is an autoimmune disease.

133. The method of any one of claims 88-132, wherein the autoimmune disease is a result directly or indirectly of aberrant splicing.

134. The method of any one of claims 88-133, wherein the autoimmune disease is selected from the group consisting of Type 1 diabetes, rheumatoid arthritis, psoriasis, multiple sclerosis, Systemic lupus erythematosus, Graves' disease, inflammatory bowel disease, Addison's disease, Sjögren's syndrome, Hashimoto's thyroiditis, Myasthenia gravis, celiac disease, Autoimmune vasculitis, Pernicious anemia, and Dermatomyositis.

135. The method of any one of claims 88-134, wherein the medical condition is a neurodegenerative disease.

136. The method of any one of claims 88-135, wherein the neurodegenerative disease is selected from the group consisting of amyotrophic lateral sclerosis, multiple sclerosis, Parkinson's disease, Alzheimer's disease, Huntington's disease, and prion diseases.

137. The method of any one of claims 88-136, wherein the medical condition is an infectious disease.

138. The method of any one of claims 88-137, wherein the infectious disease is at least bacterial, viral, fungal, or parasitic.

139. The method of any one of claims 88-138, wherein the infectious disease is selected from the group consisting of adenovirus, alphavirus, calicivirus, coronavirus (including SARS CoV2 and SARS CoV), distemper virus, Ebola virus, enterovirus, flavivirus, hepatitis virus, herpesvirus (including herpes simplex virus or varicella zoster virus), infectious peritonitis virus, influenza virus, leukemia virus, Marburg virus, orthomyxovirus, papilloma virus, parainfluenza virus, paramyxovirus, parvovirus, pestivirus, picorna virus, pox virus, rabies virus, reovirus, retrovirus, and rotavirus, Specific viruses include at least human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), and adenovirus.

140. The method of any one of claims 88-139, wherein the pharmaceutical composition further comprises a pharmaceutically acceptable carrier.

141. The method of any one of claims 88-140, wherein the pharmaceutically acceptable carrier is selected from the group consisting of solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and any combinations thereof.

142. The method of any one of claims 88-141, wherein the pharmaceutically composition is administered in solid, liquid or aerosol form.

143. The method of any one of claims 88-142, wherein the pharmaceutically composition needs to be sterile for administration as injection.

144. The method of any one of claims 88-143, wherein the pharmaceutically composition is administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by any combinations thereof.

145. The method of any one of claims 88-144, wherein the subject is a human.
